(19) United States
(12) Patent Application Publication
(10) Pub. No.: US 2024/0398291 A1
(43) Pub. Date: Dec. 5, 2024

(54) APPLICATORS FOR GLUCOSE MONITORS AND METHODS FOR APPLYING GLUCOSE MONITORS

(71) Applicant: LAXMI THERAPEUTIC DEVICES, INC., Goleta, CA (US)

(72) Inventor: Matthew Yavorsky, Granada Hills, CA (US)

(21) Appl. No.: 18/731,076

(22) Filed: May 31, 2024

Related U.S. Application Data
(60) Provisional application No. 63/505,938, filed on Jun. 2, 2023.

(51) Int. Cl. A61B 5/145; A61B 5/00

(52) U.S. Cl. CPC A61B 5/14546; A61B 5/6848

(57) ABSTRACT

A method of applying a portion of an analyte monitor to a patient using an applicator assembly includes adhering the portion of the analyte monitor to an application site while the portion of the analyte monitor is connected to an applicator assembly, and actuating the applicator assembly to advance a needle carrying at least a sensing region of a sensor member towards the desired application site. The applicator assembly is configured to release the connection between the portion of the analyte monitor from the applicator assembly prior to the tip of the needle reaching the desired application site. After the tip of the needle punctures the desired application site, the sensor member is configured to be connected to and remain held by the portion of the analyte monitor at the desired application site while the needle is configured to retract away from the desired application site.

### CROSS-REFERENCE TO RELATED APPLICATION(S)

The present application claims priority to and the benefit of U.S. Provisional Patent Application No. 63/505,938, filed Jun. 2, 2023, the contents of which are hereby incorporated by reference in their entirety.

### BACKGROUND

### Field

The present disclosure relates to applicators and other devices for facilitating attachment or otherwise applying of monitors to a body of a subject, where the monitors may measure analytes, such as glucose, in the body of the subject. The present disclosure further relates to methods of using such applicators for applying monitors.

### Description of Related Art

Monitoring different analytes in the human body can used for various diagnostic reasons. In particular, monitoring glucose levels is important for individuals suffering from type 1 or type 2 diabetes. People with type 1 diabetes are unable to produce insulin or produce very little insulin, while people with type 2 diabetes are resistant to the effects of insulin. Insulin is a hormone produced by the pancreas that helps regulate the flow of blood glucose from the bloodstream into the cells in the body where it can be used as a fuel. Without insulin, blood glucose can build up in the blood and lead to various symptoms and complications, including fatigue, frequent infections, cardiovascular disease, nerve damage, kidney damage, eye damage, and other issues. Individuals with type 1 or type 2 diabetes need to monitor their glucose levels in order to avoid these symptoms and complications.

Analyte monitors, and in particular, glucose monitors for the monitoring of glucose levels for the management of diabetes, are constantly being developed and improved. Although there are several platforms for monitoring analytes such as glucose available on the market, there is still a need to improve their precision, wearability, and accessibility to end-users. In addition, there is a desire to provide robust applicator tools that can help facilitate easier, less painful, less error-prone, and/or generally more effective application of glucose monitors to a patient's body, and in particular, continuous glucose monitors which may be attached to the patient's body for a more prolonged period of time, as well as glucose monitor features that can be used together with such improved applicators and applicator designs.

### SUMMARY

Many continuous glucose monitors are intended to be worn on a patient's skin for a duration of multiple days or weeks. Most or all commercially available glucose sensors on the market today sense glucose in interstitial fluid (ISF) below the surface of the skin. Such sensing or monitoring therefore typically involves an initial step of inserting a sensor of the glucose monitor under the patient's skin. For the most part, this insertion step will involve puncturing the surface of the skin with a needle to provide access for inserting the sensor. Applicators will generally provide a means for inserting the sensor under the patient's skin via needle or other method. In addition, continuous glucose monitors may include a device body that remains adhered to the patient for a prolonged period of time as well. Applicators may also help facilitate proper attachment of the monitor body to the patient in a manner where the sensor is properly inserted and remains in place over the duration the monitor is intended to remain functional.

An aspect of one or more embodiments of the present disclosure is directed towards an applicator assembly for applying at least a portion of an analyte monitor, where the applicator assembly includes a housing having a first end, an opposite second end, and a longitudinal axis extending between the first and second ends, an engagement portion at the second end configured to releasably engage at least part of the analyte monitor, a resilient member positioned at least partially in the housing and having at least a first portion fixed relative to the housing and a second portion movable relative to the housing in a direction substantially parallel to the longitudinal axis, a needle movable together with the second portion of the resilient member, and an actuator. When the actuator is actuated, the second portion of the resilient member and the needle are advanced towards the second end of the housing. When a tip of the needle is advanced to a first position located axially between the first and second ends of the housing, the engagement portion is adjusted from a first configuration where a portion of an analyte monitor connected to the applicator assembly is engaged by the engagement portion to hold the portion of the analyte monitor to the applicator assembly, to a second configuration where the engagement between the engagement portion and the portion of the analyte monitor is released. The tip of the needle is configured to be further advanced to a second position located axially past the second end of the housing after the engagement portion is adjusted to the second configuration and the portion of the analyte monitor is released.

The engagement portion may include a latch, where the latch may be configured to engage the portion of the analyte monitor in the first configuration and to release the engagement with the portion of the analyte monitor in the second configuration.

The applicator assembly may further include a plunger member configured to adjust the engagement portion from the first configuration to the second configuration. The plunger member may be movable together with the second portion of the resilient member and the needle towards the second end of the housing. When the plunger member, the second portion of the resilient member, and the needle are moved towards the second end of the housing, the plunger member may adjust the engagement portion from the first configuration to the second configuration prior to the tip of the needle reaching the second end of the housing. The plunger member may be configured to urge the second portion of the resilient member and the needle towards the second end of the housing. When the tip of the needle is advanced to the second position, the second portion of the resilient member and the needle may be configured to move back towards the first end of the housing, while an axial position of the plunger member remains the same.

At least part of the second end of the housing may extend substantially in a plane. The longitudinal axis may be arranged to be angled relative to the plane. The angle may be between 25° and 35°.

When the actuator is actuated, the tip of the needle may be configured to advance continuously through the first position to the second position.

The actuator may have an abutment that blocks advancement of the second portion of the resilient member and the needle prior to actuation.

When the portion of the analyte monitor is connected to the applicator assembly, the portion of the analyte monitor may be positionable between the actuator and a desired application site, such that actuation of the actuator presses the portion of the analyte monitor against the desired application site.

The portion of the analyte monitor may include a cradle or base of a two-part analyte monitor.

The second portion of the resilient member may be further configured to connect a sensor member that is separate from the portion of the analyte monitor to the portion of the analyte monitor when the tip of the needle is advanced to the second position. A sensing portion of the sensor member may be configured to be positionable in the needle, such that when the tip of the needle is advanced to the second position, the needle may be configured to puncture a patient's skin, and the sensor member may be configured to be delivered with the needle to a position under a surface of the patient's skin. The needle may be configured to be retracted away from the patient's skin after the tip of the needle reaches the second position, while the sensing portion of the sensor member may be configured to separate from the needle and remain at the position under the surface of the patient's skin.

An aspect of one or more embodiments of the present disclosure is directed towards a method of applying a portion of an analyte monitor to a patient including adhering the portion of the analyte monitor to a desired application site while the portion of the analyte monitor is connected to an applicator assembly, and actuating the applicator assembly to advance a needle carrying at least a sensing region of a sensor member towards the desired application site. The applicator assembly is configured to release the connection between the portion of the analyte monitor from the applicator assembly prior to the tip of the needle reaching the desired application site. The tip of the needle is configured to puncture the desired application site after the connection between the portion of the analyte monitor and the applicator assembly has been released. The sensor member is configured to be connected to and remain held by the portion of the analyte monitor at the desired application site while the needle is configured to retract away from the desired application site.

The applicator assembly may include a latch configured to engage the portion of the analyte monitor to connect the portion of the analyte monitor to the applicator assembly. The latch may be configured to disengage from the portion of the analyte monitor when the connection between the portion of the analyte monitor and the applicator assembly is released. The applicator assembly may further include a plunger member configured to be advanced together with the needle upon actuation. The plunger member may be configured to disengage the latch from the portion of the analyte monitor prior to the tip of the needle reaching the desired application site.

The actuating of the applicator assembly may be configured to initiate the release of the connection between the portion of the analyte monitor from the applicator assembly.

### BRIEF DESCRIPTION OF THE DRAWINGS

Further features and advantages of the invention will become apparent from the description of embodiments by means of the accompanying drawings. In the drawings:

FIGS. 1A and 1B schematically show a human body with a monitor including an analyte sensor according to embodiments of the invention, where the monitor is attached at different positions on the body.

FIG. 2 shows a perspective view from above an exemplary monitor including an analyte sensor according to embodiments of the invention.

FIG. 3 shows a perspective from below the monitor of FIG. 2.

FIG. 4A shows a perspective view from above a cradle of an exemplary monitor, for example, the monitor of FIGS. 2 and 3.

FIG. 4B shows a perspective view from above a transmitter of an exemplary monitor, for example, the monitor of FIGS. 2 and 3.

FIG. 5 shows a side view of another exemplary monitor including an analyte sensor according to embodiments of the invention.

FIG. 6A shows a perspective view from above a cradle of another exemplary monitor including an analyte sensor according to embodiments of the invention.

FIG. 6B shows a front view of the cradle of FIG. 6A.

FIG. 6C shows a side view of the cradle of FIGS. 6A and 6B.

FIG. 6D shows a top view of the cradle of FIGS. 6A to 6C.

FIG. 6E shows a cross-sectional view of the cradle of FIGS. 6A to 6D, the cross-section taken along the plane A-A shown in FIG. 6D.

FIG. 7 shows a perspective view of an applicator assembly according to a first embodiment, in a shipping or similar state prior to use by an end user.

FIG. 8A shows a cross-sectional view of the applicator assembly of FIG. 7 in the shipping state.

FIG. 8B shows a close-up of a portion of the applicator assembly shown in FIG. 8A.

FIG. 9A shows a side view of a striker or plunger of the applicator assembly in an unbiased state according to the first embodiment.

FIG. 9B shows the striker or plunger of FIG. 9A in a biased state.

FIG. 10 shows a needle carrier of the applicator assembly according to the first embodiment.

FIG. 11 shows a cap of the applicator assembly according to the first embodiment.

FIG. 12 shows a cross-sectional view of the applicator assembly according to the first embodiment after a step of cocking or otherwise biasing the striker or plunger.

FIG. 13 shows a cross-sectional view of the applicator assembly according to the first embodiment with the striker or plunger in a biased state and with the cap removed.

FIG. 14A shows a cross-sectional view of the applicator assembly according to the first embodiment with the striker or plunger in the biased state and with a button of the applicator assembly being depressed.

FIG. 14B shows a cross-sectional view of the applicator assembly according to the first embodiment in the state shown in FIG. 14A, with the cross-sectional view rotated relative to the cross-sectional view shown in FIG. 14A.

FIG. 14C shows a close-up of a portion of the applicator assembly shown in FIG. 14B.

FIG. 15 shows a cross-sectional view of the applicator assembly according to the first embodiment after the button is depressed and the striker or plunger is released such that a needle of a needle carrier of the applicator assembly is advanced and deployed for insertion into a patient's body.

FIG. 16A shows a cross-sectional view of the applicator assembly according to the first embodiment after deployment of the needle, where the striker or plunger is further advanced to conceal the needle from the outside of the applicator assembly.

FIG. 16B shows a cross-sectional view of the applicator assembly according to the first embodiment in the state shown in FIG. 16A, with the cross-sectional view rotated relative to the cross-sectional view shown in FIG. 16A.

FIG. 17 shows a perspective view of an applicator assembly according to a second embodiment.

FIG. 18A shows a perspective view of an applicator assembly according to a third embodiment, in a shipping or similar state prior to use by an end user, and with an outer housing of the applicator assembly removed to make inner components visible.

FIG. 18B shows a cutaway view of the applicator assembly of FIG. 18A in the shipping state, with the cut taken vertically through the applicator assembly.

FIG. 18C shows a cutaway view of the applicator assembly of FIGS. 18A and 18B in the shipping state, with the cut taken horizontally through the applicator assembly.

FIG. 19A shows a top view of a needle carrier of the applicator assembly of FIGS. 18A to 18C, holding a sensor member for the cradle of the analyte sensor shown in FIGS. 6A to 6E.

FIG. 19B shows a side view of the needle carrier of FIG. 19A.

FIG. 19C shows a cross-sectional view of the needle carrier of FIGS. 19A and 19B, the cross-section taken along plane B-B shown in FIG. 19A.

FIG. 19D shows an enlarged view of a portion of the needle carrier with the attached sensor member identified in FIG. 19A.

FIG. 19E shows a perspective view of the portion of the needle carrier with the attached sensor member shown in FIG. 19D.

FIG. 20A shows a top view of an exemplary needle carrier of an applicator assembly in an unbiased state.

FIG. 20B shows a top view of the needle carrier shown in FIG. 20A in a biased state.

FIG. 21A shows a perspective view of the applicator assembly according to the third embodiment during actuation, when an attached cradle has been released from the applicator assembly but prior to a needle of a needle carrier of the applicator assembly being inserted into a patient's body.

FIG. 21B shows a cutaway view of the applicator assembly in the state shown in FIG. 21A, with the cut taken vertically through the applicator assembly.

FIG. 21C shows a cutaway view of the applicator assembly in the state shown in FIGS. 21A and 21B, with the cut taken horizontally through the applicator assembly.

FIG. 22A shows a perspective view of the applicator assembly according to the third embodiment during actuation, after the needle of the needle carrier of the applicator assembly has been inserted into the patient's body.

FIG. 22B shows a cutaway view of the applicator assembly in the state shown in FIG. 22A, with the cut taken horizontally through the applicator assembly.

FIG. 23A shows a perspective view of the applicator assembly according to the third embodiment, with the needle at least partially retracted after deployment.

FIG. 23B shows a cutaway view of the applicator assembly in the state shown in FIG. 23A, with the cut taken horizontally through the applicator assembly.

FIG. 24A shows a perspective view of the applicator assembly according to the third embodiment, after the applicator assembly has been pulled away from the cradle, with the cradle adhered to a patient's skin and the sensor member inserted into the patient's skin.

FIG. 24B shows a cutaway view of the applicator assembly in the state shown in FIG. 24A, with the cut taken vertically through the applicator assembly.

FIG. 25A shows a perspective view of an applicator assembly according to a fourth embodiment, in a shipping or similar state prior to use by an end user, and with an outer housing of the applicator assembly removed to make inner components visible.

FIG. 25B shows a side view of the applicator assembly of FIG. 25A in the shipping state.

FIG. 25C shows a front view of the applicator assembly of FIGS. 25A and 25B in the shipping state.

FIG. 25D shows a cross-sectional view of the applicator assembly of FIGS. 25A to 25C in the shipping state, the cross-section taken along a plane C-C shown in FIG. 25C.

FIG. 26 shows a cross-sectional view of the applicator assembly according to the fourth embodiment, after an actuator button has been depressed.

FIG. 27 shows a cross-sectional view of the applicator assembly according to the fourth embodiment during actuation, when an attached cradle has been released from the applicator assembly but prior to a needle of a needle carrier of the applicator assembly being inserted into a patient's body.

FIG. 28 shows a cross-sectional view of the applicator assembly according to the fourth embodiment during actuation, after the needle of the needle carrier of the applicator assembly has been inserted into the patient's body.

FIG. 29 shows a cross-sectional view of the applicator assembly according to the fourth embodiment, with the needle at least partially retracted after deployment.

FIG. 30 shows a cross-sectional view of the applicator assembly according to the fourth embodiment, after the applicator assembly has been pulled away from the cradle, with the cradle adhered to a patient's skin and the sensor member inserted into the patient's skin.

### DETAILED DESCRIPTION

In the following detailed description, only certain embodiments of the subject matter of the present disclosure are described, by way of illustration. As those skilled in the art would recognize, the subject matter of the present disclosure may be embodied in many different forms and should not be construed as being limited to the embodiments set forth herein.

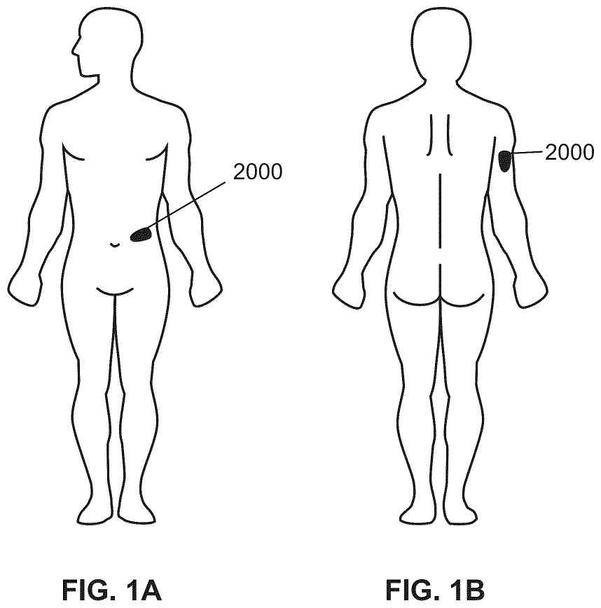

Monitors that include analyte sensors, such as glucose monitors, and particularly continuous glucose monitors, can be attached to a patient's body in different locations, in order to for example, improve glucose monitoring and/or a patient's comfort, since the continuous glucose monitors must remain adhered to the patient's skin, sometimes for a few days or more. FIG. 1A shows a first exemplary analyte monitor 2000 that is adhered to a patient's abdominal region, while FIG. 1B instead shows the exemplary analyte monitor 2000 adhered to a patient's arm. These are only meant to be example adhesion sites, and in other situations, this or a similar analyte monitor may instead be adhered or otherwise attached to other parts of the patient's body.

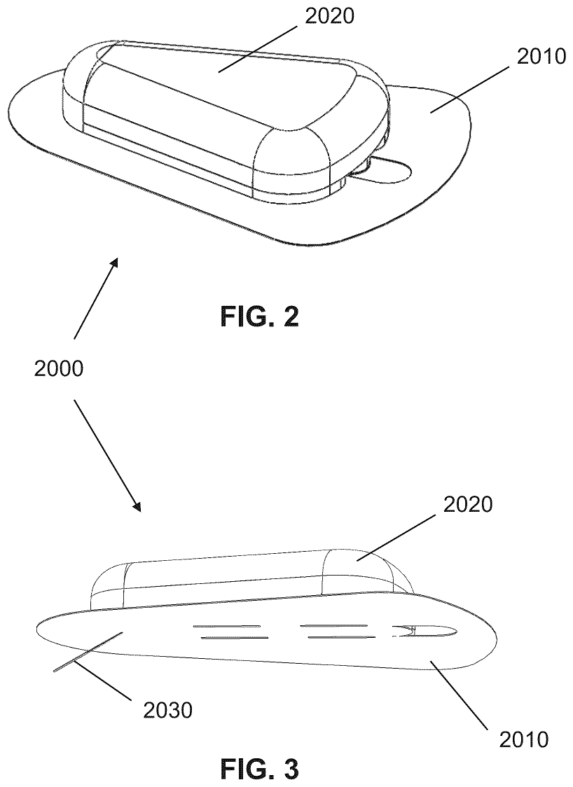

FIGS. 2 and 3 show different schematic views of an exemplary analyte monitor 2000, which can be a continuous glucose monitor, according to an embodiment of the invention. The continuous glucose monitor 2000 may include a base or cradle 2010 that may have an adhesive layer for adhering to a patient's skin, a transmitter 2020 for transmitting data to and/or from a location away from the monitor, and a sensor member 2030 which may include an integrated analyte sensing region such as a glucose sensor.

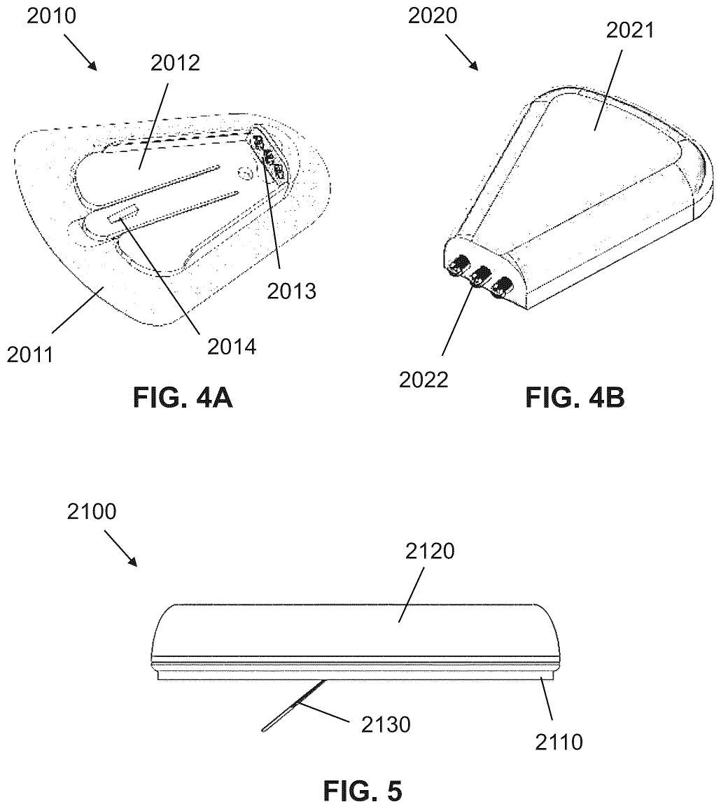

FIG. 4A shows the base or cradle 2010 of the monitor 2000. In the embodiment shown, the base or cradle 2010 is connectable and detachable from the transmitter 2020, but in other embodiments, the monitor may be a single integrated and connected unit. The cradle 2010 includes an adhesive or similar layer 2011 for connecting the cradle to the patient's skin. The adhesive layer may have a larger footprint or size compared to the rest of the cradle in order to facilitate a stronger connection to a patient or one that will last for a longer period of time, for example, multiple days or weeks. Other methods of attachment to a patient's skin may also be contemplated within the spirit and scope of the invention.

The cradle 2010 further includes a main body 2012 configured to facilitate attachment with the transmitter 2020. In the example shown, the body 2012 of the cradle is wedge-shaped to match a similar wedge shape of the transmitter 2020, but in other embodiments, the body and the transmitter may both be other shapes. In one region of the body, the cradle 2010 may further include contacts 2013 to facilitate an electrical connection with the transmitter 2020. In the embodiment shown, the contacts 2013 are located to one side of the cradle 2010, but in other embodiments, the contacts may be included in other regions of the cradle, based on the specific design and connection characteristics of the particular monitor. The cradle 2010 may also include a snap or other locking feature 2014 to facilitate a robust connection with the transmitter 2020 and reduce or eliminate occurrences of inadvertent detachment.

While not shown in FIG. 4A due to the angle of the perspective view, the cradle 2010 may further include the sensor member 2030 as an integrated component. For example, for at least some of the embodiments of applicator assemblies discussed below, the applicator assemblies are configured to attach cradles together with integrated sensors, so that actuation of the applicator assemblies will both deploy and properly position the sensor member under the patient's skin, as well as adhere the cradle to the outside of the patient's skin, in a single user step.

The base or cradle may further include other components, for example, at least some electronic circuitry to enable interconnectivity with the transmitter, and/or a battery.

The transmitter 2020 may include a main transmitter body 2021. The transmitter body 2021 includes at least a transmitter, and may further include other components such as a circuit board, memory, or other components for the transmitter to function properly. The transmitter 2020 further includes contacts 2022 configured to engage with the contacts 2013 on the cradle 2010, in order for example, to create a closed circuit for the monitor 2000 to activate and function properly.

Like the cradle, the transmitter may also include other components, for example, a battery. It will be recognized that the specific design of the monitor will dictate arrangement of different components and whether they are found on the cradle or the transmitter. For example, if a battery is housed in the cradle, a second battery will not be needed to be housed in the transmitter.

As mentioned above, other embodiments of analyte monitors may be integrated single-piece monitors. By way of example, FIG. 5 shows a side view of another exemplary monitor 2100 including an analyte sensor according to embodiments of the invention. Monitor 2100 is a one-piece monitor which includes a base 2110 that may have an integrated adhesive layer, a body 2120 which may include some or all of the components discussed above with respect to monitor 2000, and a sensor member 2030. More or less components may be needed for proper functionality of such a one-piece monitor. For example, separate contacts may not be needed in one-piece monitor designs.

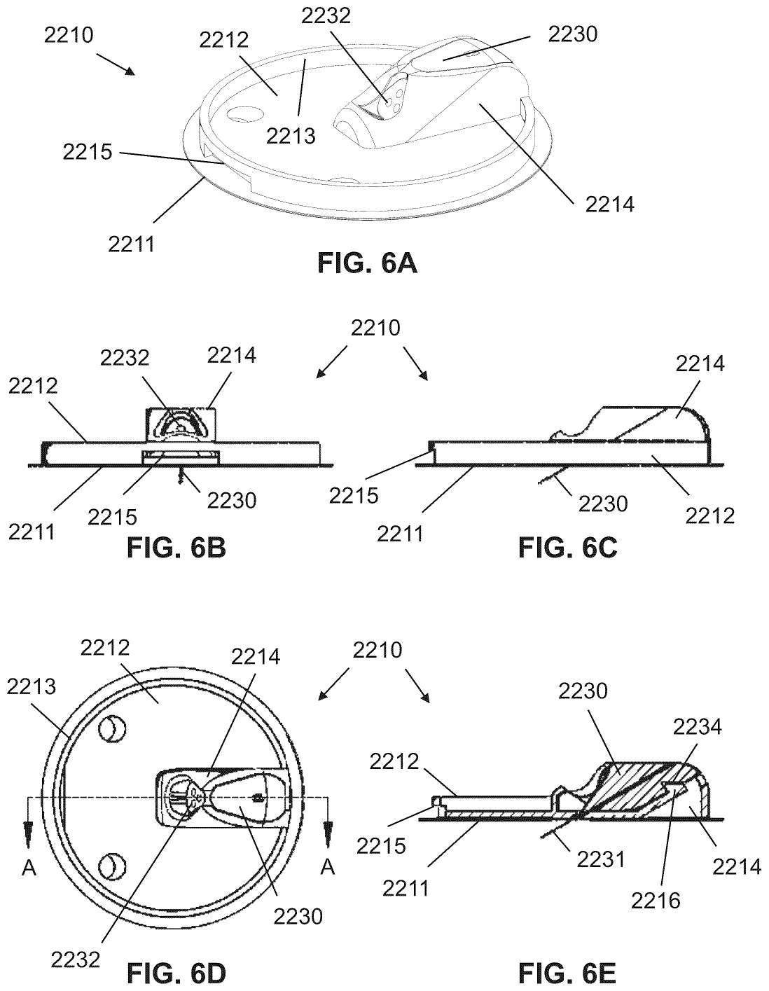

Furthermore, still other embodiments of analyte monitors may be differently shaped and have different component arrangements from the above described embodiments. FIGS. 6A to 6E show a cradle 2210 of yet another analyte monitor according to embodiments of the invention. Here, the separate transmitter of the analyte monitor is not shown, but one skilled in the art will recognize that any transmitter that is correspondingly shaped to be received in the cradle 2210 and to have contacts configured to electronically couple to the contacts described herein may be suitable for use with the cradle 2210.

Similarly to the base or cradle 2010, the base or cradle 2210 includes an adhesive or similar layer 2211 for connecting the cradle to the patient's skin. The adhesive layer may have a larger footprint or size compared to the rest of the cradle for a wider surface area, in order to facilitate a stronger connection to a patient or one that will last for a longer period of time, for example, multiple days or weeks. Other methods of attachment to a patient's skin may also be contemplated within the spirit and scope of the invention.

The cradle 2210 further includes a main body 2212 configured to facilitate attachment with a separate transmitter. In this embodiment, the body 2212 is round or circular shaped to match a similar round or circular shape of the transmitted, but in other embodiments, the body and transmitter may both be other shapes. In some cases, an asymmetric shape may be preferable over a round or circular shape, in order to more easily facilitate a blind connection with a transmitter by a user later (e.g., after the cradle has been attached to the patient's skin on a part of the body that is not visible or only partially visible to the patient, such as the back of an arm). The body 2212 may include a sidewall 2213 and/or other features configured to at least partially house the transmitter when connected, and may further include one or more attachment mechanisms to facilitate holding of the transmitter and the cradle together after connecting the parts together. Such connection features may be formed on the sidewall 2213, on another part of the body 2212, on another portion of the cradle 2210, or on any combination thereof.

The cradle 2210 in this embodiment further includes a sensor housing 2214 for housing a separate sensor member 2230. The respective shapes of the housing 2214 and sensor member 2230 may be complementary, so as to provide an at least partially form-fit connection between the parts when assembled. In exemplary embodiments of the invention, an applicator assembly may connect the cradle 2210 with the sensor member 2230 automatically when the cradle is applied to the patient's skin, together with placing and properly positioning an analyte sensor 2231 of the sensor member below a surface of the patient's skin, where the cradle 2210 will then hold the proper position of the analyte sensor 2231 after deployment, as will be described in greater detail below. As can best be seen in FIGS. 6A, 6D, and 6E, in this embodiment, the housing 2214 may have a rear opening that is large enough to facilitate insertion of the sensor member 2230 therethrough, a front opening configured to expose contacts 2232 of the sensor member 2230 when the sensor member has been fully inserted into the housing 2214, and a bottom opening configured to facilitate passage of the analyte sensor 2231 therethrough and into the skin of the patient. The housing 2214 may further include a latch 2216 or other holding mechanism (as best seen in FIG. 6E) to engage a corresponding recess or other engagement surface 2234 on the sensor member 2230 for holding the sensor member 2230 in place after insertion and to prevent the sensor member 2230 from being separated from the cradle 2210 through the back opening of the housing 2214. In some embodiments, two or more of the described openings may communicate with one another, or in other words, be formed as two parts of a continuous opening, or may be otherwise arranged, so long as the functionality described herein is properly facilitated. In some embodiments, an outer, side, and/or inner surface of the housing 2214 may further be shaped to facilitate attachment with at least part of the applicator assembly, to provide additional stability during shipping and/or deployment of the cradle onto a patient's skin.

The cradle 2210 may further include a ledge 2215 or other protrusion, recess, or other attachment mechanism, that is configured to hold the cradle 2210 to the applicator assembly prior to deployment. In this embodiment, the ledge 2215 defines a recess thereunder into which a latched connection with the applicator assembly is facilitated, described in greater detail below. However, any other attachment mechanism is feasible, so long as adequate attachment to the applicator assembly can be achieved, and release is possible via the mechanisms and methods described below.

As has been alluded to above, the sensor member 2230 includes an analyte sensor 2231 and one or more electrical contacts 2232 in communication with and configured to facilitate signal delivery of the sensor 2231 to a separate part, for example, to a processor housed in a separate transmitter that is configured to be connected to the cradle 2210. As can be seen in FIGS. 6A to 6E, the connection between the cradle 2210 and the sensor member 2230 is made by advancing the sensor member 2230 at an angle into the back opening of the housing 2214, where the sensor member 2230 then snaps into place in the housing 2214. This allows for an angulated delivery of the analyte sensor 2231 into the patient, where in the embodiment shown, the angle may be between 25° and 35°, or more broadly between 20° and 40°. However, in other embodiments, the analyte sensor may be delivered under the skin at a straight or perpendicular orientation to the surface of the patient's skin, and/or at a smaller or greater angle than that shown in the described embodiment or embodiments.

In some embodiments, either the cradle 2210 or the sensor member 2230 may further house a battery (not shown), where in such cases, a separate battery may therefore not be needed in a transmitter configured to be connected to such cradle. Regardless of where a main battery is housed in an analyte sensor, electrical connections between the respective parts can be arranged to facilitate supplying of power to all electrical components after the respective parts are connected to one another. The location of the battery can be selected to be most beneficial based on the respective design of each embodiment and objectives thereof.

As mentioned above, in some embodiments, an applicator assembly is provided for attaching either a cradle or an entire monitor to a patient's skin, where the applicator assembly may serve to both properly insert and position a sensor member under a patient's skin, as well as to properly and securely adhere the cradle or monitor body to an outer surface of the patient's skin. Preferably, placement of the sensor member and the cradle or monitor body can both be accomplished together via a single action by the user, for example, an actuation of a button on the applicator assembly.

In general, an applicator assembly that includes a needle deployment to facilitate placement of a sensor member under the skin will further include an integrate sharps protection mechanism, that will retract the needle or otherwise shield the needle from the outside of the applicator assembly once the sensor has been deployed, to avoid injuries caused by the needle being exposed to the outside of the applicator assembly.

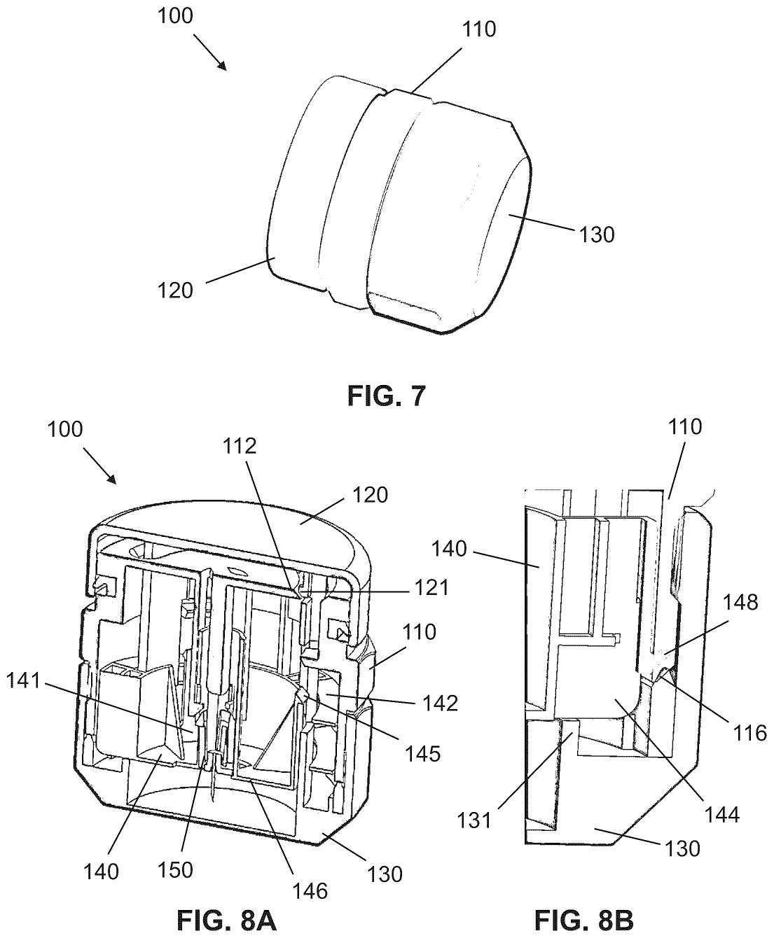

A first embodiment of an applicator assembly is shown in FIGS. 7 to 16B. The applicator assembly 100 according to the first embodiment may be a molded spring applicator, where an internal spring mechanism and/or other parts of the applicator assembly may be injection molded or otherwise molded. The applicator assembly includes a main body or frame 110, a button 120, a cap 130, a plunger or striker 140, and a needle carrier 150. More or less parts may be included in other embodiments based on their specific designs. FIGS. 7 to 8B show the applicator assembly 100 in an initial shipping or storage state. Here, the applicator assembly is generally shipped as a sterile assembly, where an elastomer or other sterile barrier that may be found on the cap or on the frame helps facilitate keeping the assembly sterile. In some instances, plastic-on-plastic engagements, or engagements other than elastomer-based engagements, may be utilized to save on costs, but may be slower or more difficult to manufacture. Other types of barriers may also be utilized. The applicator assembly 100 may further be shipped in the initial shipping state in a sterile packaging.

In the embodiment shown, the applicator assembly 100 in its initial shipping state may be approximately 6 cm in length with the cap attached, and have an outer diameter of about 6 cm as well. In other embodiments, the size and shape of the applicator assembly may be larger or smaller, and/or have another shape, based at least in part on the size and shape of the wearable device (e.g., the cradle or the one-piece monitor) to be applied to the patient.

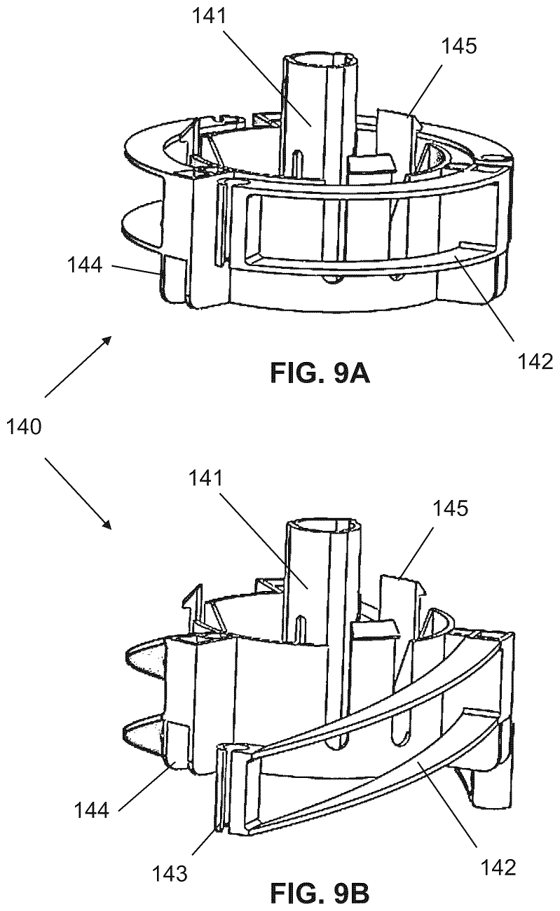

F FIGS. 9A and 9B show a striker or plunger 140 of the applicator assembly 100 according to the first embodiment. The plunger 140 forms a primary resilient member for the applicator assembly. The plunger 140 includes a central compartment 141 that may be sized and shaped to house the needle carrier 150 therein in an axially movable manner. For example, if the needle carrier has a substantially cylindrical shape or a similar shape with a circular cross-section, the central compartment 141 of the plunger 140 may similarly be shaped, for example, substantially cylindrical or conical. The plunger 140 may further include at least one resilient portion such as springs 142 to facilitate movement of the plunger 140 within the frame 110. The springs 142 in this embodiment are integrally formed with the rest of the plunger 140, and extend circumferentially around the plunger 140 to a free end 143 with an attachment means configured to attach to the frame 110. In the embodiment shown, the springs are molded, for example, injection molded, but may be formed in any other suitable manner. In addition, the springs 142 are integrally formed with the rest of the plunger 140, but in other embodiments may be separate parts that are connectable to the rest of the plunger 140. Here, the embodiment includes three springs, each extending slightly less than 120° around the circumference of the plunger 140, such that the three springs together extend around substantially an entire circumference of the plunger 140. In other embodiments, more or less springs may be included based on the desired strength and movement desired of the plunger 140 relative to the frame 110 to effectively deploy the needle and/or other components for delivering the wearable device. And in still other embodiments, other advancement mechanisms besides springs can also be used. At an end of the springs 142 opposite the free ends 143, the plunger 140 includes engagement portions 144 that are configured to move along ramps of the cap 130 in order to bias the springs, as will be described in greater detail below.

FIG. 9A shows the plunger 140 in an unbiased state, where the springs 142 are unbiased. FIG. 9B shows the plunger 140 in a biased or fully cocked state, where a preload is applied, for example, to the main body of the plunger 140, while the ends 143 of the springs 142 are held in place axially, resulting in the springs 142 biasing in an axial direction.

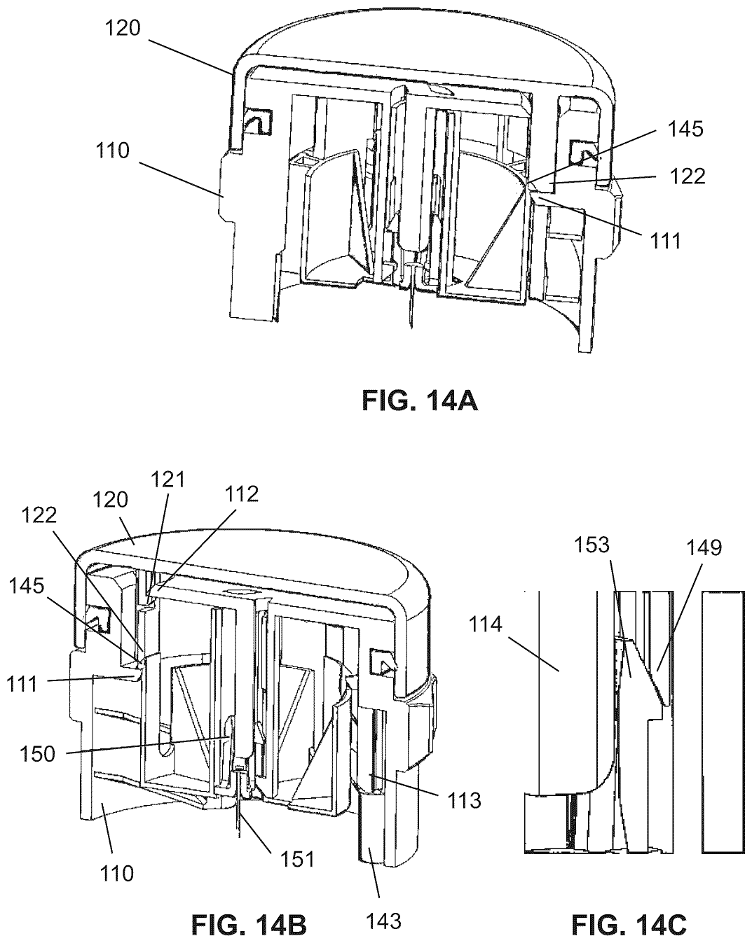

At or near an upper end of the plunger 140, latches 145 may further be included, where the latches may be flexible, for example, may deflect radially inwardly. Furthermore, as labeled in FIG. 8A, a bottom surface 146 of the plunger 140 may be sized and shaped to effectively receive and hold a wearable device, for example, a cradle of a continuous glucose monitor, where downward advancement of the plunger 140 results in downward advancement of the wearable device as well, until the wearable device abuts against the patient's skin. For wearable devices where an adhesive is present, the adhesive will cause the wearable device to attach to and remain on the patient's skin, while the applicator assembly can be removed and detached from the wearable device. As best seen in FIG. 13, the bottom of the plunger 140 may also form an inner ledge 147, which limits downward or distal movement of the needle carrier 150 relative to the plunger 140, such that when the needle carrier 150 abuts against the ledge 147, the needle 151 may extend out of the bottom of the rest of the applicator assembly 100 a desired amount. In some embodiments, the plunger 140 may further include additional features, such as latches 148 (see, e.g., FIG. 8B) and posts 149 (see, e.g., FIG. 14C), which help facilitate operation of the applicator assembly 100, and which will be described in greater detail below.

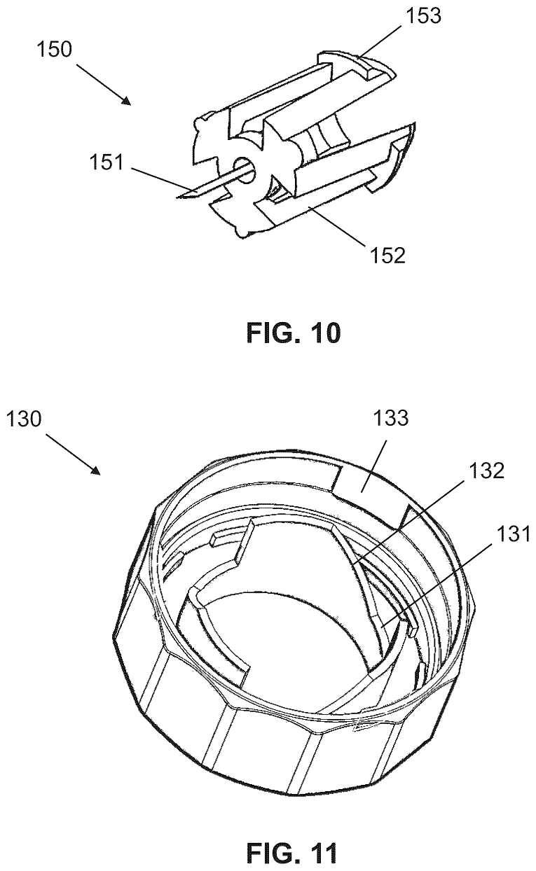

A needle carrier 150 according to the first embodiment of the applicator assembly 100 is shown in FIG. 10. The needle carrier 150 includes a needle 151 that may be attached to a body 152. The carrier body in this embodiment may have a substantially circular outer profile, with stops or abutments 153 at one side thereof that are formed on ends of axially extending walls. The walls of the needle carrier may further be flexible, for example, radially inwardly and/or outwardly, such that the stops 153 are radially movable, for example, depending on the stroke position. The needle carrier should not be limited to the disclosed embodiment, and other needle carrier arrangements may also be used instead, based on the overall design of the applicator assembly.

A cap 130 according to the first embodiment of the applicator assembly 100 is shown in FIG. 11. The cap 130 includes a circular track that extends around an inner chamber of the cap. The circular track may include shelves 131 and ramps 132, where the shelves 131 may extend parallel to a bottom surface of the cap 130, while the ramps 132 extend in an inclined manner from the shelves 131 towards the opening of the cap. In this embodiment, there are three shelves 131 and three ramps 132, to correspond to the three springs 142 of the plunger 140. However, in other embodiments, more or less shelves and/or ramps may be included based on the design of the applicator assembly, and in particular, based on the arrangement of the springs included in the applicator assembly. The cap 130 may further include a projection 133 or other form of engagement feature configured to engage a corresponding engagement feature on the frame (not shown). For example, in this embodiment, the projection 133 on cap 130 is rectangular in shape, and the cap 130 may include three similar projections 133 spaced equidistantly around the cap, for example, in 120° intervals. The frame may include an interrupted thread or track, for example, a track that extends slightly less than 120°, so that rotation of the cap 130 relative to the frame 110 over the length of the track will allow disengagement of the cap 130 from the frame 110. In other embodiments, the interrupted thread or track may be on the cap, and similar projections as those shown on the cap in this embodiment may be provided on the frame instead. In other embodiments, the track may be inclined or otherwise arranged to facilitate rotation of more than 120° even with three projections, for example, a 150° rotation to effect disengagement. Or any other arrangement where disengagement of the cap from the frame with less than a full 360° rotation of the cap is possible can be used. Allowing for detachment of the cap from the frame with less than a full 360° rotation may be beneficial to minimize or reduce relative movement between the parts and simplify detachment for the end user. In addition, detachment without excessive rotation may be needed due to the design of the ramps 132, which may make full 360° rotation of the cap difficult, as described in greater detail below.

During manufacturing, the cap 130 may be designed as a push-to-connect part, for example, where the cap 130 and/or the frame 110 may be slightly flexible, and/or via one-way ramps built into the structure of the parts, to facilitate easier connection of the parts together. However, axial disconnection of the parts may be undesirable, in order to properly operate the device and prevent user error, for example. Therefore, it should only be possible to disconnect the cap 130 from the frame 110 in this embodiment by rotation of the cap 130 and the frame 110 relative to one another. In another assembly method, the parts may simply be assembled the same way they are intended to be disassembled. For example, in this embodiment where removal of the cap requires approximately a 120° rotation of the cap 130 relative to the frame 110, assembly may simply require a simple bayonet action, where a quick engagement and rotation between the parts completes assembly. In yet other embodiments, other connection mechanisms can also be utilized.

Referring back to FIGS. 7 to 8B, in the initial shipping or storage state, the springs 142 may be completely unbiased, or may be biased only a small amount. A slight preload from the time of manufacture may be desirable, so long as there is not enough load to cause creep of the parts. In addition, referring specifically to FIG. 8B, a slight preload on the springs 142 may ensure that plunger 140 is positioned slightly axially higher relative to frame 110, which may prevent latches 148 from unintentionally engaging ledge 116 on the frame 110. Latches 148 are intended to engage ledge 116 after firing of the applicator assembly, in order to act as a lock to prevent retraction of the plunger 140 back into the frame 110, so that the plunger 140 can be used as a sharps guard as described in greater detail below. In some embodiments, an undercut may be included to house the latches 148 in the shipping state, to prevent a preload acting on the latches 148. In other embodiments, the springs may be biased or preloaded a greater amount, for example, fully biased in a “ready to fire” state, at manufacture.

As previously mentioned, a sterile barrier may be formed between the cap and the frame to seal the interior of the applicator assembly and keep it sterile. In addition, a similar seal may be formed between the button and the frame for the same purpose. Furthermore, the cap or another portion of the assembly may include a safety mechanism to prevent the button from inadvertently misfiring before the wearable device is ready to be deployed.

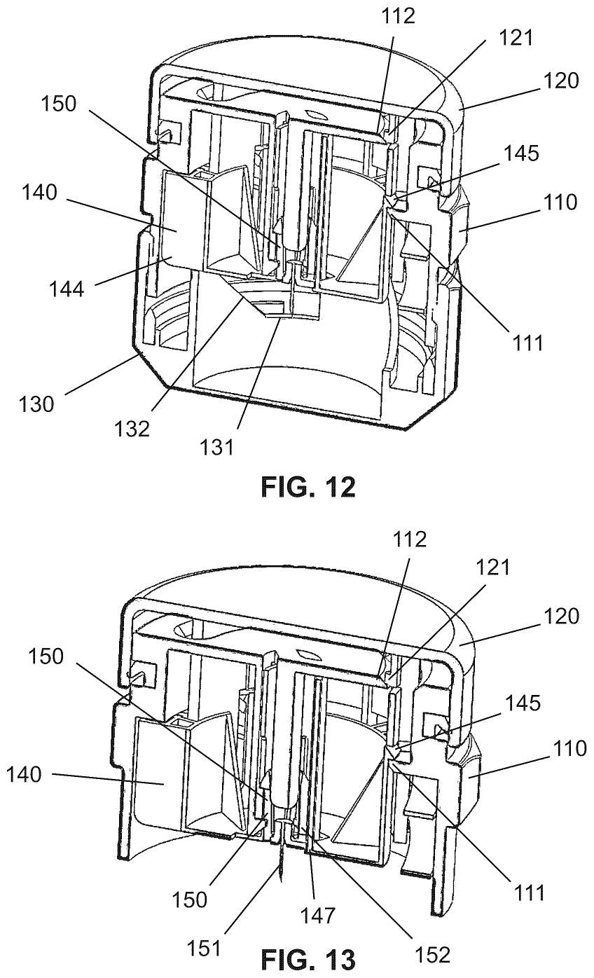

FIGS. 12 and 13 illustrate the applicator assembly 100 in a ready-to-fire state. Prior to cocking of the applicator assembly, as best seen in FIGS. 8A and 8B, the engagement portions 144 of the plunger 140 rest on the shelves 131 of the cap 130. Referring then to FIGS. 11 and 12, an unscrewing or otherwise rotation of the cap 130 relative to the frame 110 will cause the engagement portions 144 of the plunger to move up the ramps 132, e.g., in a proximal direction towards the button 120, while the ends 143 of the springs 142 are held in the same axial position relative to the frame 110 by engagements 113 (see, e.g., FIG. 14B). This results in a biasing of the springs 142 in the manner shown in FIG. 9B, such that the base of the plunger 140 is held at the higher position shown in FIG. 11. The plunger 140 is moved upwards until latches 145 snap over or otherwise engage ledge 111 of frame 110 to hold the body of the plunger 140 at this higher cocked position. At the same time, the needle carrier 150 is also moved upwards together with the body of the plunger 140 via the engagement at the ledge 147. At the fully cocked position shown in FIGS. 12 and 13, the free end of the needle will be axially recessed from a lower or distal end of the frame 110, such that the needle will not extend axially out of the applicator assembly 100 until the button 120 is depressed. Rotation of the cap until the plunger 140 is at the fully cocked position may coincide with the projections 133 of the cap 130 aligning with the openings on the tracks of the frame 110, to allow for removal of the cap 130 from the rest of the applicator assembly 100 only when the plunger 140 has been fully cocked and latches 145 have snapped into place. After removal of the cap 130, as shown in FIG. 13, the applicator assembly is ready for use to apply an attached wearable device (not shown).

Loading the applicator assembly 100 via rotational cocking provides a number of benefits over pre-loaded springs. For example, storing a plastic spring in a cocked state for prolonged periods of time may degrade the spring, as cold flow and creep can negatively affect performance of the spring. Having the user cock the spring while removing a sterile barrier cap eliminates the cold flow risk by only cocking the plastic firing spring at the time of use. Furthermore, integrating the spring cocking into the barrier removal process makes the cocking invisible to a user, and may also provide a mechanical advantage to achieve higher cocking forces for higher velocity needle insertion.

Steps of firing the applicator assembly 100 are shown in FIGS. 14A to 16B. Actuation or firing of applicator assembly 100 is designed as a one-step process for the end user, for example, by depression of button 120 relative to frame 110. In practice, after removal of cap 130, the user places the applicator assembly 100 at the desired location of attachment of the cradle or other wearable device, with the open end facing towards the skin. Once a desired deployment location is selected, the button 120 is depressed towards the skin, which results in both the firing of the applicator assembly 100 to place the wearable device on the skin and the sensor under the surface of the skin, and the deployment of the sharps guard thereafter to retract and/or otherwise conceal the needle 151 for safety purposes.

Referring first to FIGS. 14A to 14C, when button 120 is depressed, posts 122 of button 120 are moved downward relative to frame 110 until they engage latches 145 of plunger 140. A bottom surface of posts 122 may serve to push latches 145 radially inwardly to make latches 145 disengage from ledges 111 of frame 100. One or both of the top of latches 145 and/or the bottom of posts 122 may be tapered or inclined to facilitate the radially inward movement of the latches 145. The button 120 itself may only move down a small fixed axial distance, and another latch, for example, latch 121 on button 120, may engage ledge 112 of frame 110 to hold the button 120 at its lower depressed position, in order to ensure deployment of the plunger 140. In some embodiments, engagement of the latch or latches 121 with the ledge 112 may result in an audio or tactile click to provide feedback to the user that the button has been successfully fully depressed. In some embodiments, the latches 121 may differ in number and size, for example, may be longer or shorter than shown in the figures, depending on the specific design of the applicator assembly.

When the latches 145 of plunger 140 are urged inwardly and disengage from ledge 111 of frame 100, the body of the plunger 140 is no longer held in the upward position, and the bias of springs 142 serve to urge the body of plunger 140 downwards. The downward movement of the plunger 140 will also result in the downward movement of the wearable device or other payload that is connected to the bottom 146 of the plunger 140 towards the user's skin. As best seen in FIGS. 14B and 14C, the needle carrier 150 including the needle 151 are also urged downward together with the plunger 140. The walls of the needle carrier 150, including the latches 153, are blocked from deflecting radially inwardly by a post 114 on the frame 100, and additional posts 149 on the plunger push down on the tops of latches 153, so that plunger 140 and needle carrier 150 translate downwardly together towards the user's skin. Due to the presence of post 114, even if the tops of latches 153 are inclined, the downward pressure applied by posts 149 of plunger 140 will result in downward movement of the needle carrier 150 because the post 114 prevents inward movement of the latches 153 to escape the downward force of posts 149.

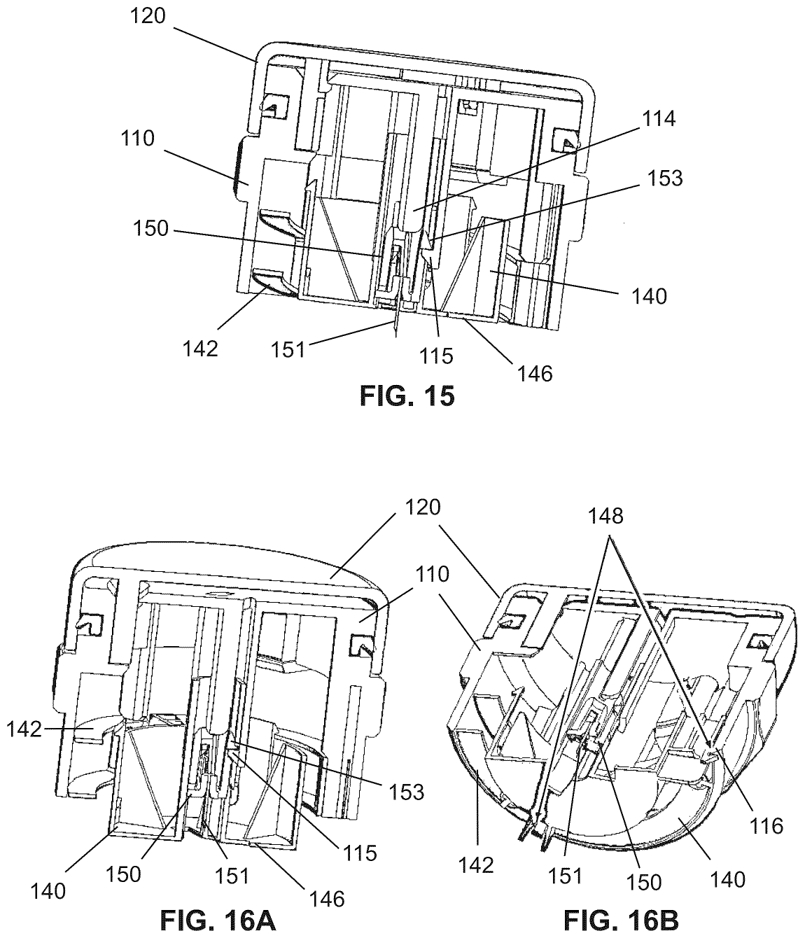

In FIG. 15, the needle carrier 150 has reached its lowermost position during the deployment process. In this position, the bottom 146 of the plunger 140 will urge the wearable device or other payload against the user's skin, while the needle 151 and any loaded sensor member is pushed through the skin to a desired position under the surface of the skin. The length of the needle 151 will change depending on the size and configuration of the applicator assembly, the desired depth and positioning of the sensor member, and on the size and shape of the wearable device or other payload, for example, the shape of the cradle to be delivered together with the sensor member, and how the payload is loaded and held by the applicator assembly. In the embodiment shown, the needle is inserted perpendicularly to the user's skin surface into the skin. In other embodiments, such as some discussed below, the needle may be inserted into the user's skin at an angle relative to the surface of the skin. Here, further downward movement of the needle carrier is prevented due to engagement of the latches 153 on the needle carrier 150 with the ledge 115 of the frame 110. At this height, the walls of the needle carrier 150 may also be axially clear of the post 114, which will allow the walls to deflect inward slightly in order to allow the posts 149 of the plunger 140 to pass by them, thereby allowing the plunger 140 to continue to move downwards relative to the frame 110 while the axial position of the needle carrier 150 relative to the frame 110 remains the same.

After the downward movement of the needle carrier 150 relative to the frame 110 is stopped, the bias on the springs 142 will continue to force the plunger 140 downwards relative to the frame 110 and the needle 151 without any additional user input. Since the bottom 146 of the plunger 140 is already pressed against the user's skin, or against the payload which is held against the user's skin, additional movement of the plunger 140 relative to the frame 110 results in pushing of the frame 110 and the needle 151 away from the user's skin, effectively pulling the needle out of the user's body. Furthermore, since the needle 151 is retracted axially relative to the plunger 140, the body of the plunger 140 acts as a sharps guard to shroud or surround the needle 151 once the plunger 140 is fully advanced relative to the frame 110 and the needle 151, as shown in FIGS. 16A and 16B. In this position, the needle 151 is safely housed within the plunger 140 after deployment. Here, the springs 142 may be able to return to or close to their unbiased states, where latches 148 snap under or otherwise engage ledges 116 at or near a bottom end of the frame 116, to prevent the plunger 140 from moving back into the frame 110. Since the positions of both the plunger 140 and the needle 151 are fixed relative to the frame 110 in this final position, the needle 151 is prevented from being exposed to the outside of the applicator assembly. Here, the cap 130 can be replaced over the bottom of the applicator assembly for additional protection and disposal.

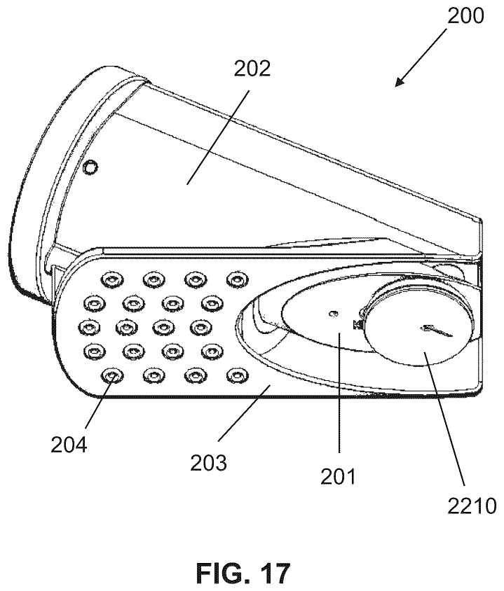

A second embodiment of an applicator assembly is shown in FIG. 17. Parts, portions, and operations of the second embodiment of the applicator assembly that are similar to the first embodiment will not be repeated.

Rather than deploying a needle in a perpendicular direction to the surface of a patient's skin, the applicator assembly 200 in the second embodiment is configured to deploy a needle at an angle to the patient's skin. The applicator assembly 200 in the second embodiment may have an engagement surface 201 that includes one or more connection mechanisms or means for connecting and holding an analyte monitor or a portion of an analyte monitor such as cradle 2210 discussed in connection with FIGS. 6A to 6E prior to deployment. The applicator assembly 200 may further include an angled main housing 202 in which various parts for deploying a needle and for detaching the cradle 2210 therefrom may be housed. Similarly as described above, a preferred angular arrangement of the housing may allow for deployment of the needle at a 25° to 35° angle to the surface of the skin, more broadly at a 20° to 40° angle to the surface of the skin, or at any of various other angles based on the requirements of the sensor being deployed, among other factors.

To facilitate an angled deployment, the internal components of the applicator assembly 200 may also be arranged at an angle, and may include all or some of the mechanisms described above with respect to applicator assembly 100 according to the first embodiment, and may further include additional internal parts and features in order to facilitate an angular operation.

While not pictured in this embodiment, the applicator assembly further includes a button or other actuator to initiate deployment. Upon actuation of the actuator, the needle assembly will fire to facilitate implanting of the analyte sensor at a desired depth and angle, and will in some embodiments further retract in a single step, so as to remove the needle from the patient while the cradle and the analyte sensor remain in place relative to the patient.

In addition, the applicator assembly 200 may also include a bottom engagement surface 203 configured to rest on the patient's skin. The bottom engagement surface 203 may be arranged at a desired angle of deployment relative to the main housing 202, so as to help facilitate positioning of the applicator assembly 200 at a proper angle during actuation or deployment, for example, when the entire engagement surface 203 or at least a majority of the engagement surface 203 is properly pushed against the user's skin prior to deployment. To this effect, the engagement surface 203 may be formed relatively large relative to the rest of the applicator assembly 200, as illustrated. In other embodiments, the engagement surface may be formed larger or smaller than pictured, or may be omitted altogether if another feature is included to facilitate proper angulation of the applicator assembly by the patient.

The bottom engagement surface 203 may further include one or more high friction elements 204, such as added texture or beads made of high friction material, so that when held firmly against the user's skin, the applicator assembly 200 can be held in place more steadily and not slide around. This may improve usability of applicator assemblies with angled or steep insertion angles, where a force from firing may tend to otherwise shift the applicator assembly during application of a given payload. Other friction elements and/or mechanisms can also be implemented in other embodiments with similar results. In one embodiment, the high friction surface 204 may further assist in pulling the skin at the insertion site taut or bunch the skin up as the user slides the applicator assembly as part of an applicator process or workflow. In another embodiment, the user can slide the applicator and create tension at the high friction surface that can overcome a spring force to release the actuator and deploy the sensor, for example, in lieu of the buttons previously discussed in earlier embodiments. Other implementations of high friction surfaces may also be utilized in still other embodiments.

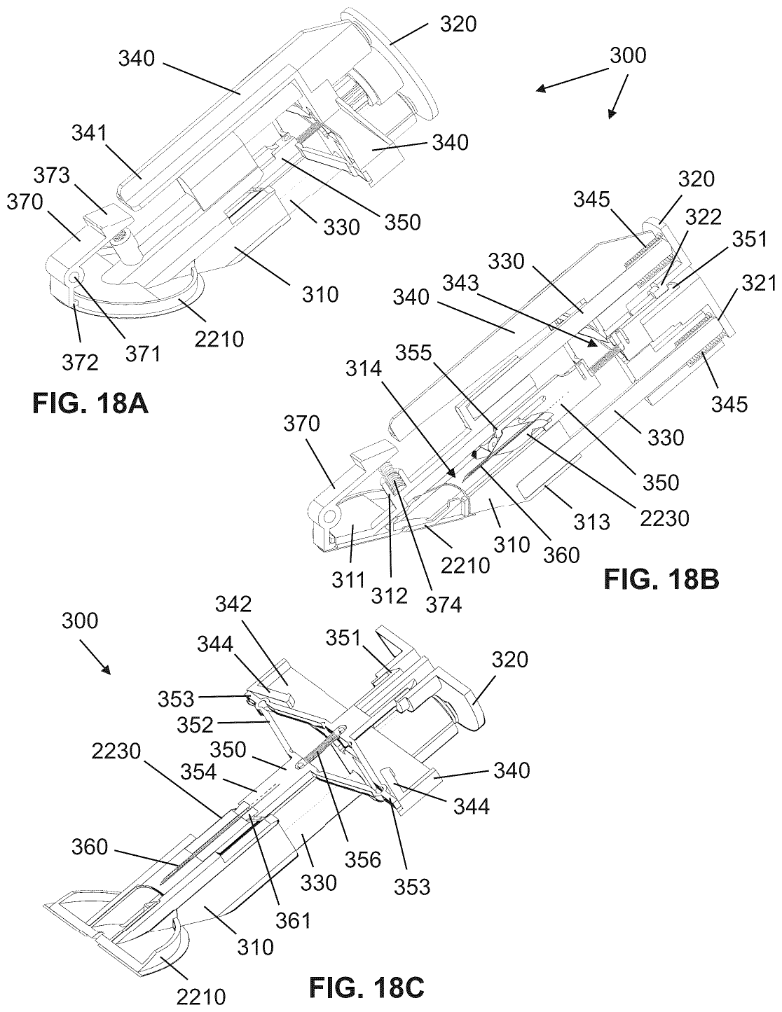

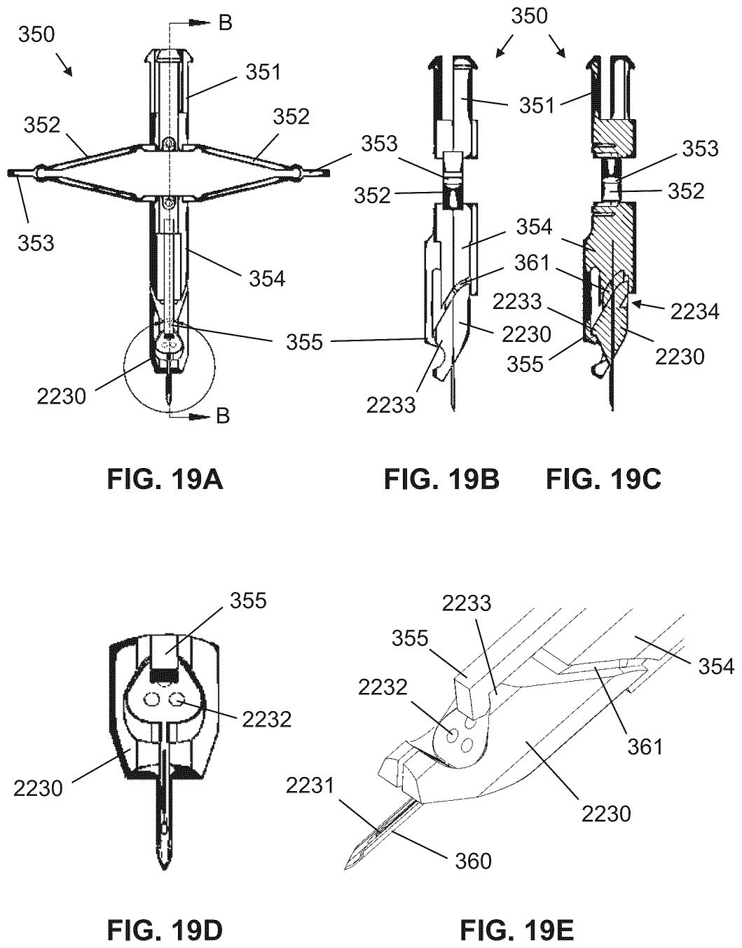

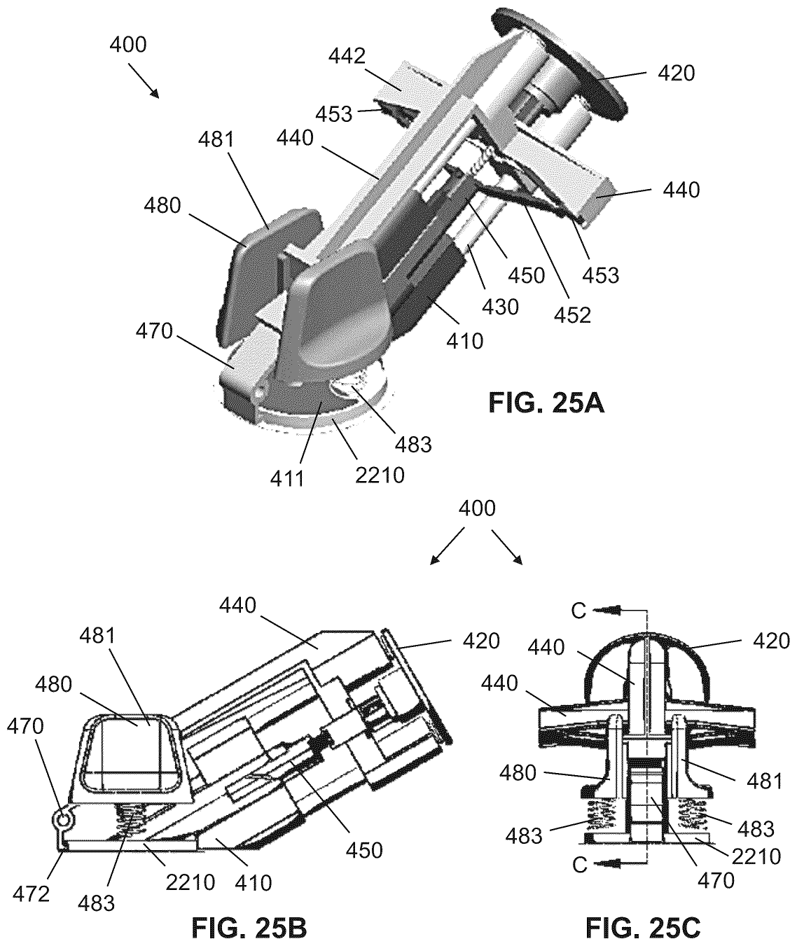

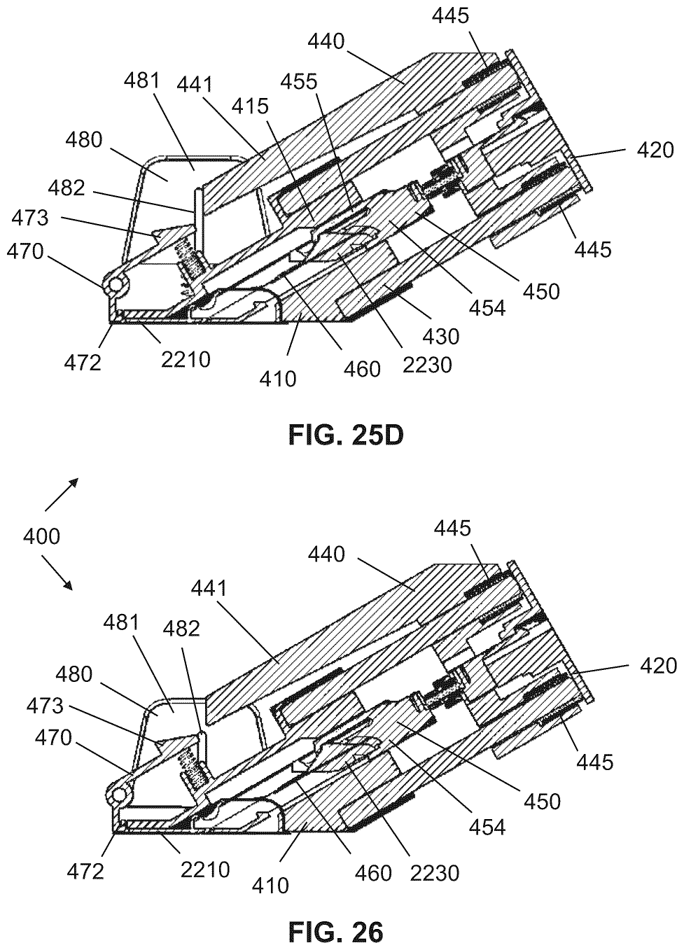

A third embodiment of an applicator assembly is shown in FIGS. 18A to 24B. Parts, portions, and operations of the third embodiment of the applicator assembly that are similar to the first and second embodiments will not be repeated. Here, the applicator assembly 300 according to the third embodiment is being presented with a housing of the applicator assembly removed or omitted, in order to visualize and discuss the internal components of the applicator assembly more clearly. As can be gleaned from the figures, the applicator assembly 300 according to the third embodiment is configured to deploy a needle and insert an analyte sensor into a patient's skin at an angle, for example, 25° to 35° relative to the surface of the skin, or at another angle as appropriate. The internal components of the applicator assembly 300 can be housed, for example, in the housing 210 shown with respect to the applicator assembly 200 of the second embodiment, but may also be housed in a housing of a different applicator assembly, so long as an angle of deployment the selected housing is configured for matches the angle of deployment of the internal components. In addition to omission of the housing, an actuator or trigger for activating and initiating deployment of the applicator assembly 300 has not been illustrated. It should be understood that a button or trigger similarly as previously discussed, or any other type actuator, can be integrated into the design without departing from the spirit or scope of the invention. One such actuator is further described in the fourth embodiment of the applicator assembly shown in FIGS. 25A to 30, and discussed in greater detail below.

FIGS. 18A to 18C show internal components of the actuator assembly 300 in an initial state, for example, a shipping state or a state where the actuator assembly is cocked and ready for firing/deployment. As can be seen, a body or cradle 2210 of an analyte monitor as discussed with reference to FIGS. 6A to 6E, or any other suitable cradle or entire analyte monitor, may be packaged together with the actuator assembly 300, or for example, may be manually attached by a user prior to deployment in some cases.

Referring to FIGS. 18A to 18C, the actuator assembly 300 includes a first distal fixed portion 310 whose position is fixed relative to the housing, and a second proximal fixed portion 320 whose position is also fixed relative to the housing. In some embodiments where the first and second fixed portions 310, 320 are spaced apart from one another, additional support posts 330 may extend between the first and second fixed portions 310, 320, to help maintain a constant structure of the applicator assembly 300.

Examining the first distal fixed portion 310 in greater detail, the first fixed portion includes a first engagement portion 311 configured to engage the cradle 2210, another suitable cradle, or an entire analyte monitor intended to be applied to a patient's skin. In the embodiment shown, the first engagement portion 311 includes at least a rounded region configured to occupy at least part of the internal space of the cradle 2210, to minimize or eliminate movement between the parts when connected. The first engagement portion 311 may further include one or more connecting means that either connect to the frame of the cradle 2210, or more specifically to a part of the housing 2214 for the sensor member 2230, for example, an outer surface thereof. The first engagement portion 311 may further attach or stabilize against the cradle 2210 in any of various other ways. The first fixed portion 310 may further include a recess or other portion 312 configured to connect to a spring 374 or other biasing mechanism of a latch 370 or other connecting member that itself is configured to releasably hold the cradle 2210 to the applicator assembly 300. In the embodiment shown, the connector 312 is formed by a bore into which the spring 374 extends and is fixed. For embodiments that include support posts 330, the first fixed portion 310 may further include a region 313 for connecting to the support posts 330, which in the embodiment shown are a series of recesses into which the support posts extend. The first fixed portion 310 and the support posts may be fixed relative to one another by, for example, a glue or other adhesive, threading, interference fit, or any other suitable method. In addition, an angled bore 314 extends through the first fixed portion. The angled bore is sized and configured to facilitate passing through of a sensor member 2230 and at least part of a delivery part, for example, a distal portion 354 of a needle carrier 350 that is configured to hold and deliver the sensor member 2230 to the cradle 2210.

The second proximal fixed portion 320 may be arranged as a proximal end plate, or in some embodiments may include additional features. The second fixed portion 320 may serve as a seal or other closing feature that engages the housing to prevent access to the internal components of the applicator assembly 300. In the embodiment shown, the second proximal fixed portion 320 includes at least an abutment surface 321 against which support posts 330, springs 345 of a plunger member 340, or both, and/or additional components may abut. The second fixed portion 320 may further include attachment means for fixedly connecting, for example, proximal ends of the support posts 330, proximal ends of the springs 345, or both, to for example facilitate structural stability with the support posts 330, and/or to provide a fixation point to allow springs 345 to expand, as will be described in greater detail below. The second fixed portion 320 may further include an attachment feature, for example, a ledge 322, for engaging and holding a proximal end 351 of the needle carrier 350 stationary relative to the housing of the applicator assembly 300. In other embodiments, attachment of a portion of the needle carrier 350 to the second fixed portion 320 or another part of the applicator assembly 300 may be realized in other ways.

The applicator assembly may further include a plunger member 340. The plunger member 340 is movable relative to the housing and the fixed portions of the applicator assembly 340, and in particular, is movable axially along an axis of deployment of a needle 360. The plunger member 340 in this embodiment is a solid member, but may be flexible and/or otherwise manipulable in other embodiments. The plunger member 340 includes a plunger or post 341, wings 342 configured to engage the needle carrier 350, and springs 345 or other biasing member configured to launch or otherwise deploy the plunger member 340 distally relative to the fixed portions of the applicator assembly 300. The post 341 may be an elongate member that generally extends substantially parallel to the axis of deployment of the needle 360 and that is axially aligned along its axis of extension with the latch 370. The wings 342 are configured to engage and push at least part of the needle carrier 350, as described in greater detail below. The wings 342 may define a central bore 343 therebetween to house at least part of the needle carrier 350, for example, part of the proximal end 351 so that the proximal end 351 can engage the second fixed portion 320, and may further include abutting surfaces 344 that are configured abut against outer tabs 353 on the needle carrier 350 and to push and/or otherwise facilitate advancement of part of the needle carrier 350, as described in greater detail below. The springs 345 may be biased and compressed during shipping, whereby energy is stored in the springs 345 prior to actuation, and the springs 345 may serve to fire or launch the plunger member 340 in a generally distal direction upon release of a catch via actuation of a button, trigger, or other actuation mechanism. The proximal ends of the springs 345 may be fixed to the second fixed portion 320, such that the distal ends of the springs 345 are configured to push the plunger member 340 distally when the springs 345 expand from their compressed state. Other embodiments may include other advancement mechanisms in lieu of springs, for example push rods, where operation can be similarly carried out in such embodiments.

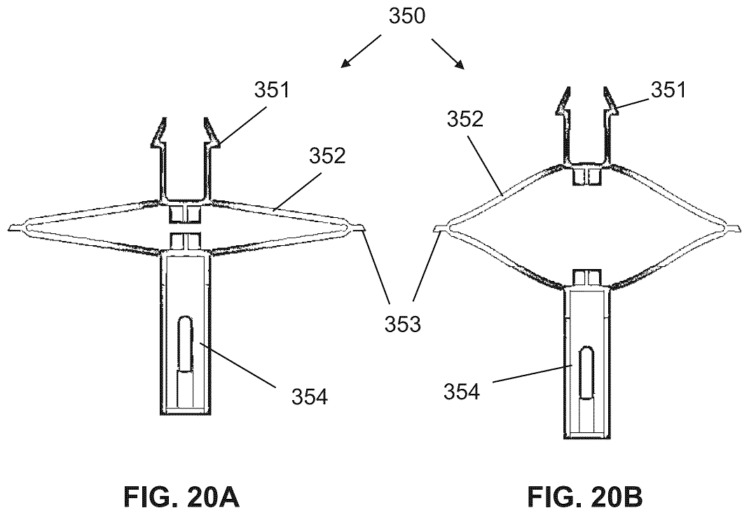

The needle hub or needle carrier 350 will now be described in greater detail, with reference to FIGS. 18A to 20B. Then needle carrier 350 includes a fixed or stationary proximal portion 351, which may include tabs or other engagement means for engaging the fixed second portion 320 or another stationary portion of the applicator assembly 300. In the embodiment shown, the tabs of proximal portion 351 extend and snap into the second portion 320, such that when a distal portion of the needle carrier 350 moves distally, the needle carrier 350 stretches axially and an axial length of the needle carrier 350 is configured to increase accordingly. The needle carrier further includes resilient arms 352 connected at one end to the proximal portion 351, and connected at an opposite end to a movable distal main body 354. The resilient arms 352 are arranged such that the axial ends 351, 354 of the needle carrier 350 are urged back towards one another by the resilient arms 352 if they are pulled away from one another, for example, by outside forces. In other words, when the main body 354 of the needle carrier 350 is pulled away axially from the proximal end 351, the resilient arms 352 are pulled apart axially and urged inwardly radially, and are thereafter configured to return towards their original shape to effectively urge the main body 354 back towards the proximal end 351 (see, e.g., FIGS. 20A and 20B). In this manner, the needle carrier 350 can be considered to be constructed similarly to a rivet member. To further facilitate snapback, the needle carrier 350 may further include an integrated spring 356 or other biasing member to pull the main body 354 back towards the proximal end 351 after deployment. In the embodiment shown, the spring 356 is connected directly between the portions 351, 354, to more effectively pull the two part towards one another when stretched out axially. At radially outward positions on the resilient arms 352 are arranged substantially flat and solid tabs 353 that extend radially outwardly and that are configured to engage the wings 342 of the plunger member 340, as discussed in greater detail below. The main body 354 of the needle carrier 350 is configured to engage and hold the sensor member 2230 in a releasable manner, and may include for example, a snap or latch 355 or other securing means configured to engage a corresponding ridge or recess 2233 on the sensor member 2230. The main body 354 of the needle carrier 350 is further connected to the needle 360, which extends distally therefrom.

The needle 360 is substantially hollow, to facilitate holding of the analyte sensor 2231 therein when the sensor member 2230 is attached to the needle carrier 350. The needle 360 is further of sufficient length to extend completely through the sensor member 2230, and to extend out of a distal end thereof, so that the distal tip of the needle 360 is able to puncture the patient's skin and implant the analyte sensor 2231 to a desired position under the patient's skin. The sensor member 2230 may include a passage extending therethrough to facilitate passing of the needle 360 therethrough. The needle 360 may further include, at a proximal end where the needle 360 is connected to the needle carrier 350, a compressible or otherwise compliant pad or other member 361, which may for example be spongy, positionable between the needle carrier 350 and the sensor member 2230. The compliant member 361 may be used to cushion or otherwise soften delivery of the sensor member 2230 into the cradle 2210, which may for example, reduce the likelihood of damage to the respective parts when the cradle assembly is assembled upon deployment of the applicator assembly 300. In some embodiments, the compliant member 361 may actuate a mechanism to break or otherwise separate the needle 360 from the sensor member 2230 when the pad 361 is compressed, and/or to ensure advancement of the analyte sensor 2231 of the sensor member 2230 to a desired depth before the needle 360 begins retracting away from the sensor member 2230.

The latch 370 may be a rotatable latch that rotates around a pivot point, for example, pivot 371. The pivot 371 may be attached, for example, to the first fixed portion 310, to the housing, or to another fixed or stationary part of the applicator assembly 300. As shown in FIGS. 18A and 18B, the latch 370 in a shipping or other initial state, may include a projection 372 that is configured to engage the cradle 2210 to provide a releasable hold of the cradle against separating from the applicator assembly 300. At an opposite end of the latch 370, an engagement portion is configured to engage the post 341 of the plunger member 340, and may include, for example, a ramped surface 373 or other surface that facilitates rotation of the latch 370 relative to the pivot 371 when the post 341 engages the surface 373. Upon engagement between the ramped surface 373 and the post 341, the latch 370 may rotate in a manner such that the projection 372 moves away from and releases the hold on the cradle 2210. A catch spring or other biasing member 374 may be attached to the first fixed portion 310, and may serve to hold the latch 370 in a latched position where the latch 370 engages the cradle 2210, prior to the post 341 engaging and pushing down on the ramped surface 373 of the latch 370 to release the hold on the cradle 2210.

Operation of the applicator assembly 300 according to the third embodiment will now be described with reference to FIGS. 18A to 24B. As discussed above, FIGS. 18A to 18C show the applicator assembly 300 in an initial or shipping state. If the cradle 2210 is not pre-attached to the applicator assembly 300, the patient can manually attach the cradle 2210 to the applicator assembly 300 prior to actuation of the applicator assembly 300. In cases where an adhesive patch under the base of the cradle 2210 is present, the patient may remove a cover of the adhesive patch and adhere the cradle 2210 to the patient's skin at a desired location on the patient's body.

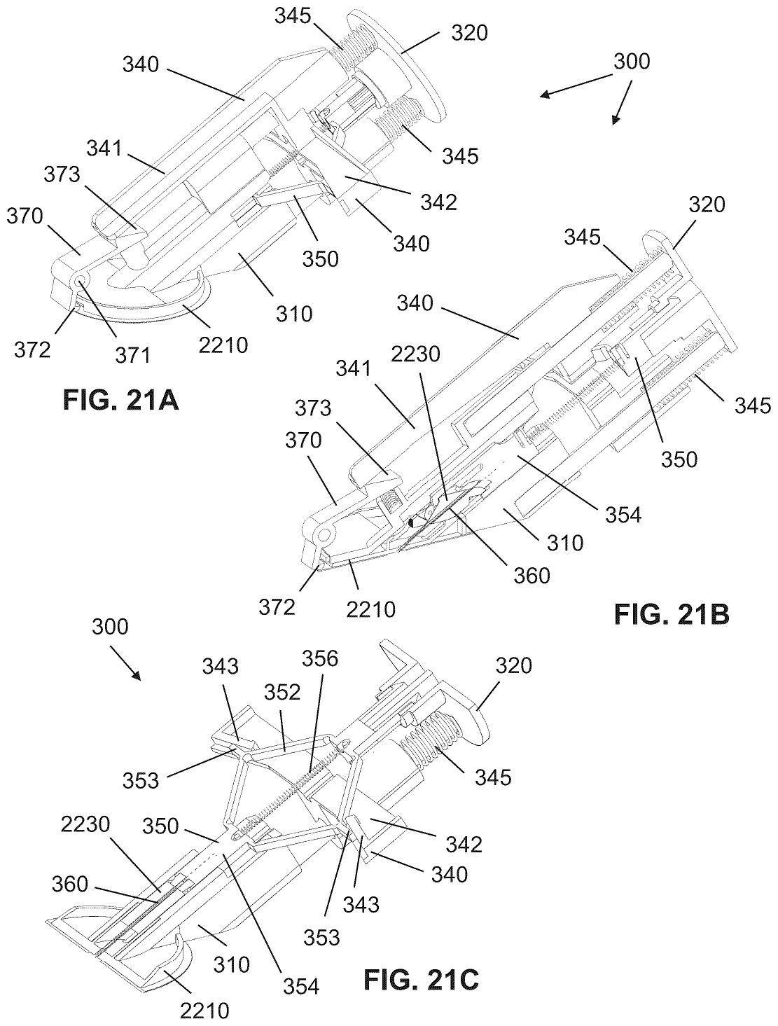

As shown in FIGS. 21A to 21C, after a trigger is actuated to release a blocking member or other type of hold on the plunger member 340 (not shown), the energy stored in the springs 345 is released, and the springs 345 begin to expand and push the plunger member 340 distally relative to the stationary portions of the applicator assembly 300. Distal movement of the wings 342 of the plunger member 340, and in particular the abutments 343, also push the main body 354 of the needle carrier 350 distally (e.g, via the arms 352 and tabs 353), which in turn accelerates and urges the needle 360 and the sensor member 2230 distally towards the cradle 2210 and the patient's skin as well. The axial stretching of the arms 352 of the needle carrier 350 also cause the arms 352 to start collapsing radially inwardly and the tabs 353 to move radially inwardly, as can best be seen in FIG. 21C. In addition, as best seen in FIGS. 21B and 21C, the extension spring 356 of the needle carrier 350 also begins stretching. In general, the resiliency of the arms 352 of the needle carrier 350 as well as the extension spring 356 may both be weaker than the firing springs 345, and in some embodiments significantly so, such that the extension of the extension spring 356 will not affect or will only minimally slow the distal advancement of the plunger member 340.

When the plunger member 340 has been moved a sufficient distance distally, the plunger member 340 causes post 341 to contact and actuate latch 370, by pushing down on surface 373, which in turn causes latch 370 to rotate about pivot 371 and projection 372 to disengage from cradle member 2210, thereby releasing the hold between the applicator assembly 300 and the cradle member 2210. Such disengagement of the applicator assembly 300 from the cradle member 2210 or other payload in this embodiment occurs before the needle 360 reaches the patient's skin, in other words, the cradle 2210 is released from the applicator assembly 300 before the analyte sensor 22310 has been inserted into the patient's skin or even before the cradle 2210 has been fully assembled. This arrangement may be beneficial in various different ways, for example, current applicators often run into issues where patients have trouble disengaging the applicators from a fully implanted cradle or monitor due to insufficient detachment mechanisms in the current designs. Early release of the cradle reduces or eliminates such situations from arising, and the early release on the connection should further allow for some wiggle room during deployment, which may further increase comfort for the patient.

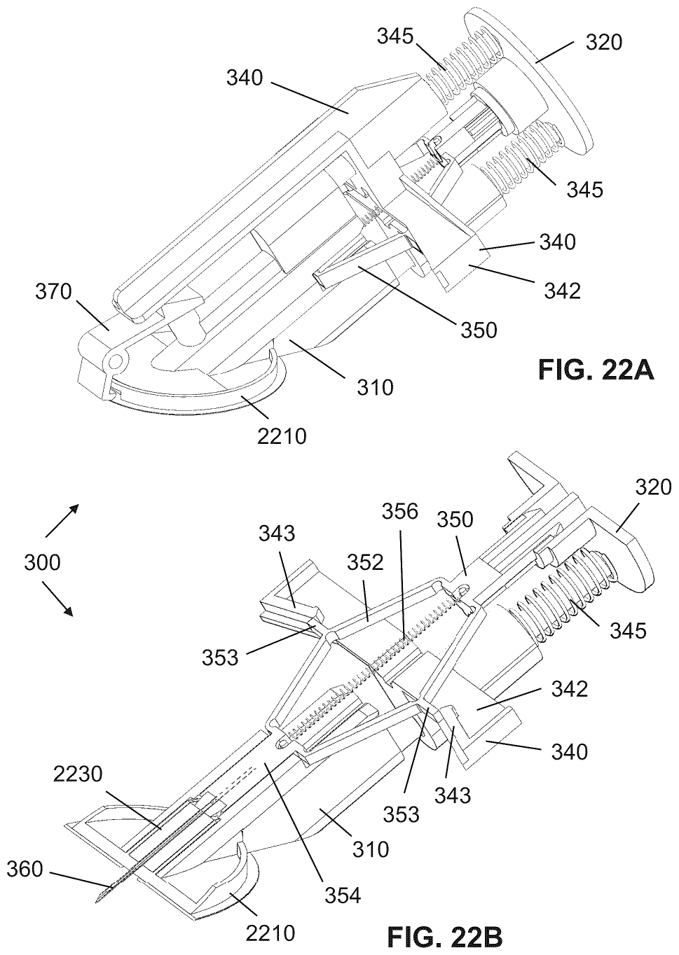

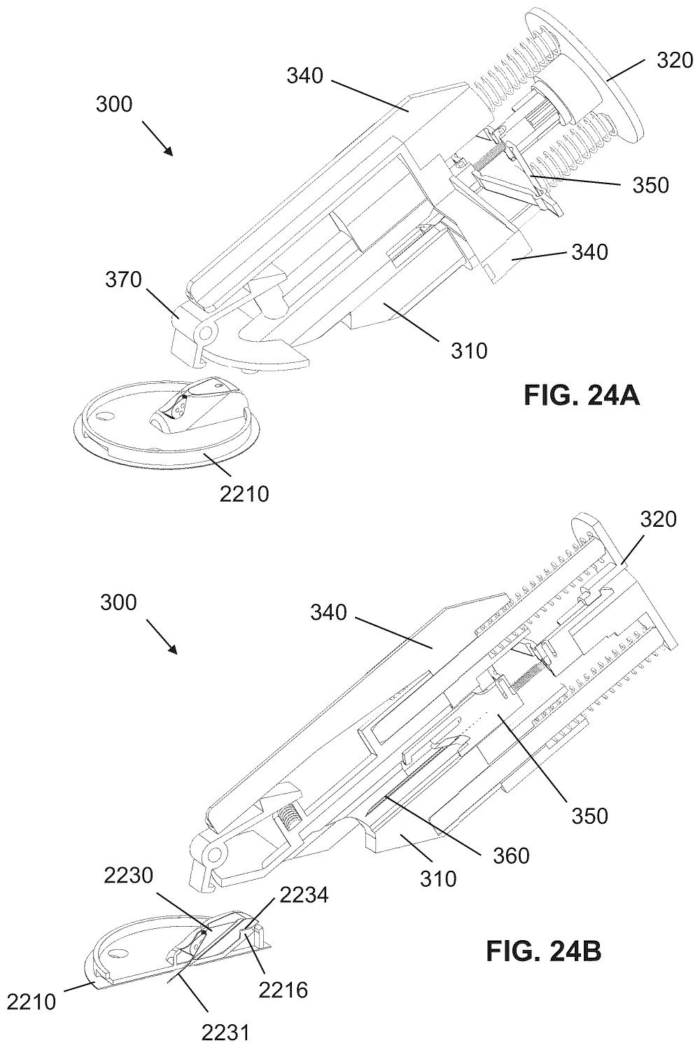

As seen in FIGS. 22A and 22B, further advancement of the plunger member 340, and in turn, the main body 354 of the needle carrier 350, results in sufficient advancement of the needle 360 and the analyte sensor 2231 housed therein to extend distally out of the distal end of the applicator assembly 300 and puncture and penetrate the skin of the patient. When the sensor member 2230 is advanced to a desired position relative to the cradle 2210, for example, to a fully assembled position between the cradle 2210 and the sensor member 2230, an engagement latch 2216 or other feature on the cradle 2210 may engage a corresponding engagement surface 2234 on the sensor member 2230 to hold the respective parts together (see, e.g., FIGS. 6E and 24B). Such engagement ensures a proper positioning of the sensor member 2230 relative to the cradle 2210, and consequently, a proper positioning of the analyte sensor 2231 relative thereto and relative to the patient as well, for example, at a desired depth and/or with a certain orientation of the sensor, for example, with the sensor chemistry facing upwards towards the surface of the skin. The snap or other engagement 2216, 2234 between the cradle 2210 and the sensor member 2230 may be more rigid or otherwise stronger than the snap feature formed by the latch 355 of the needle carrier 350 on the sensor member 2230, such that when the sensor member 2230 is finally assembled to the cradle, the stronger connection will cause the needle carrier 350 to disengage from and thereby release the sensor member 2230. At approximately the same time, the axial extension of the needle carrier 350 will be such that the arms 352 of the needle carrier 350 will have collapsed or constricted radially inwardly to an extent such that the tabs 353 will no longer abut against the abutments 343 of the wings 342 of the plunger member 340 and will be allowed to slip past them. As such, while the springs 345 will hold the plunger member 340 in place distally, the tabs 353 of the needle carrier 350 will be allowed to move proximally past the abutments 343 of the plunger member 340, and the arms 352 of the needle carrier will thereafter be allowed to contract axially and expand radially again, thereby pulling the main body 354 of the needle carrier proximally relative to the other portions of the applicator assembly 300.

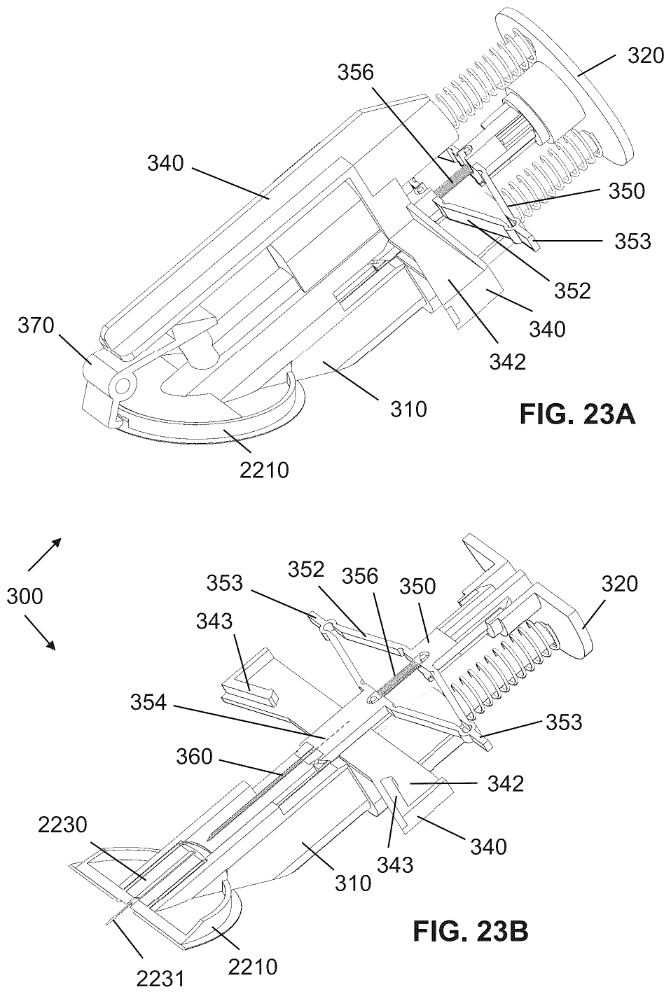

FIGS. 23A and 23B show the retraction of the main body 354 of the needle carrier 350 relative to the other parts of the applicator assembly 300 described above. While the plunger member 340 is held at a distal position, for example, by springs 345, the tabs 353 of the needle carrier 350 are allowed to slip radially inside and proximally past the abutments 343 on the plunger member 340. This allows the initial stiffness or resiliency of the arms 352, as well as the extension spring 356, to pull the main body 354 of the needle carrier 350 back proximally relative to the other parts of the applicator assembly 300. Proximal retraction of the main body further retracts needle 360 in the proximal direction as well, pulling the needle out of the patient's body and back into the housing of the applicator assembly 300, which may then serve as a sharps guard for the needle 360 after actuation and deployment. As can best be seen in FIG. 23B, while the needle 360 has been retracted, engagement between the cradle 2210 and the sensor member 2230 holds the sensor member 2230 in place relative to the cradle 2210, while the analyte sensor 2231 remains deployed at a desired location under the surface of the patient's skin. This can be facilitated, as discussed above, by the hold between the cradle 2210 and the sensor member 2230 being stronger than the hold between the sensor member 2230 and the needle carrier 350.

In a last step of the applicator assembly 300 shown in FIGS. 24A and 24B, the applicator assembly 300 is pulled away from the assembly including the cradle 2210 and the connected sensor member 2230. The cradle 2210 is held in place on the patient's skin, for example, via the adhesive on the bottom side thereof, while the analyte sensor 2231 is securely held at a desired location under the surface of the patient's skin. Thereafter, in cases where a cradle is applied by the applicator assembly, a separate transmitter or other durable or reusable portion may then be attached to the cradle, both mechanically and electrically, to complete the assembly of the analyte monitor.

A fourth embodiment of an applicator assembly is shown in FIGS. 25A to 30. Parts, portions, and operations of the fourth embodiment of the applicator assembly that are similar to the first, second, and third embodiments will not be repeated. Similar to the third embodiment, the applicator assembly 400 according to the fourth embodiment is being presented with a housing of the applicator assembly removed or omitted, in order to visualize and discuss the internal components of the applicator assembly more clearly. As can be gleaned from the figures, the applicator assembly 400 according to the fourth embodiment is configured to deploy a needle and insert an analyte sensor into a patient's skin at an angle, for example, 25° to 35° relative to the surface of the skin, or at another angle as appropriate.

In general, the applicator assembly 400 according to the fourth embodiment is very similar to the applicator assembly 300 according to the third embodiment, and parts that are identical or similar will not be repeated, while discussion will focus on the differences between the two embodiments. To this effect, a proximal second fixed portion 420 is similar to the proximal second fixed portion 320 of the third embodiment, support posts 430 are similar to the support posts 330 of the third embodiment, a plunger member 440 is similar to the plunger member 340 of the third embodiment, a needle 460 is similar to the needle assembly 360 of the third embodiment, and a latch 470 is similar to the latch 370 of the third embodiment.

Most notably, the applicator assembly 400 of the fourth embodiment differs from the applicator assembly 300 of the third embodiment by the addition of a button or other actuator 480, which facilitates actuation of the applicator assembly 400 and initiates firing and deployment of the same. The button 480 includes a main button body 481, which may include two halves on either side of the applicator assembly 400. In some embodiments, the main button body 481 may instead be a single integrated piece, or may be made up of more than two pieces. At an inner surface of the main button body 481, a blocking rib 482 is formed which blocks advancement of the post 441 of the plunger member 440, as best seen in FIG. 25D. The blocking rib 482 in this embodiment is vertical and extends laterally a sufficient amount to form an abutment that abuts against a distal end of the post 441. The button 480 may further include springs 483 or other resilient member or members that hold the button 480 in the blocking position until a user depresses or otherwise actuates the button 480. In this embodiment, the springs 483 are located at and extend downwardly from a lower end of the button 480. The lower ends of spring 483 may be attached, for example, to engagement portion 411 of the distal first fixed portion 410, and extend between the engagement portion 411 and the button 480.

Further to the above, the first fixed portion 410 may further differ from the first fixed portion 310 of the third embodiment by the addition of a stop feature 415 that prevents flexing of the latch 455 on the main body 454 of the needle carrier 450 when in the initial or shipping position (see, e.g., FIG. 25D). In this embodiment, the stop feature 415 is formed by an enlarged inner surface that reduces the size of the passageway through the first fixed portion 410, such that the enlarged inner surface abuts against the latch 455 and restricts movement of the latch 455 away from the sensor member 2230 when the sensor member 2230 is attached to the main body 454 of the needle carrier 450. In this manner, the latch 455 can more securely hold the sensor member 2230 to the main body 454 of the needle carrier 450 during shipping and/or otherwise prior to activation of the applicator assembly 400.

The needle carrier 450 according to the fourth embodiment may also be slightly different from the needle carrier 350 according to the third embodiment. While not illustrated, the needle carrier 450 may further include an additional snap or other engagement feature, which may engage upon retraction in order to hold the needle carrier 450 in the retracted state after deployment. This may be coupled with a smaller distal opening of the first fixed portion 410, in order to more securely guard the needle 460 after retraction. Here, the distal opening of the first fixed portion 410 may be sufficiently small to prevent any fingers from being inserted therein, and the catch, snap, or other feature on the needle carrier 450 may hold the needle sufficiently retracted from the distal opening of the first fixed portion 410, such that it may be impossible for a patient to inadvertently poke themselves with the biohazard needle after actuation of the applicator assembly 400.

Operation of the applicator assembly 400 according to the fourth embodiment is generally similar to operation of the applicator assembly 300 according to the third embodiment. First, as shown in an initial or shipping state in FIGS. 25A to 25D, the blocking rib 482 of the button 480 blocks distal advancement of the post 441 of the plunger member 440, while the springs 483 hold or bias the button 480 in place in an upper blocking position. With the plunger member 440 in the cocked or retracted position, the springs 445 are compressed and store energy for later firing of the plunger member 440. Here, the needle carrier 450 is also in the retracted position by virtue of the stiffness of the arms 452 and the extension spring 456. Meanwhile, the cradle 2210 is secured to the applicator assembly 400 via the projection 472 of the latch 470. In embodiments where the cradle 2210 is not preassembled to the applicator assembly 400, the patient can manually attach the cradle 2210 to the applicator assembly 400 when they are ready to apply the cradle 2210 to their skin.

In a first step, the cradle 2210 with the attached applicator assembly 400 is adhered at a desired location on the patient's skin, similarly as discussed above with respect to the third embodiment. The user then presses down on the main body 481 of the button 480, as shown in FIG. 26, which moves the blocking rib 482 downward to a position where the post 441 of the plunger member 440 is no longer blocked by the blocking rib 482 and is therefore free to advance distally relative to the rest of the applicator assembly 400. In this embodiment, the positioning of the button 480 directly above the cradle 2210 may also result in application of further downward pressure on the cradle 2210, which may serve to further secure and add to the adhesion of the cradle 2210 to the surface of the patient's skin, and/or act as a stabilizing pressure during deployment where the force applied by the patient contributes to holding the assembly in securely in place during activation of the applicator assembly 400 and deployment of the cradle 2210 therefrom.

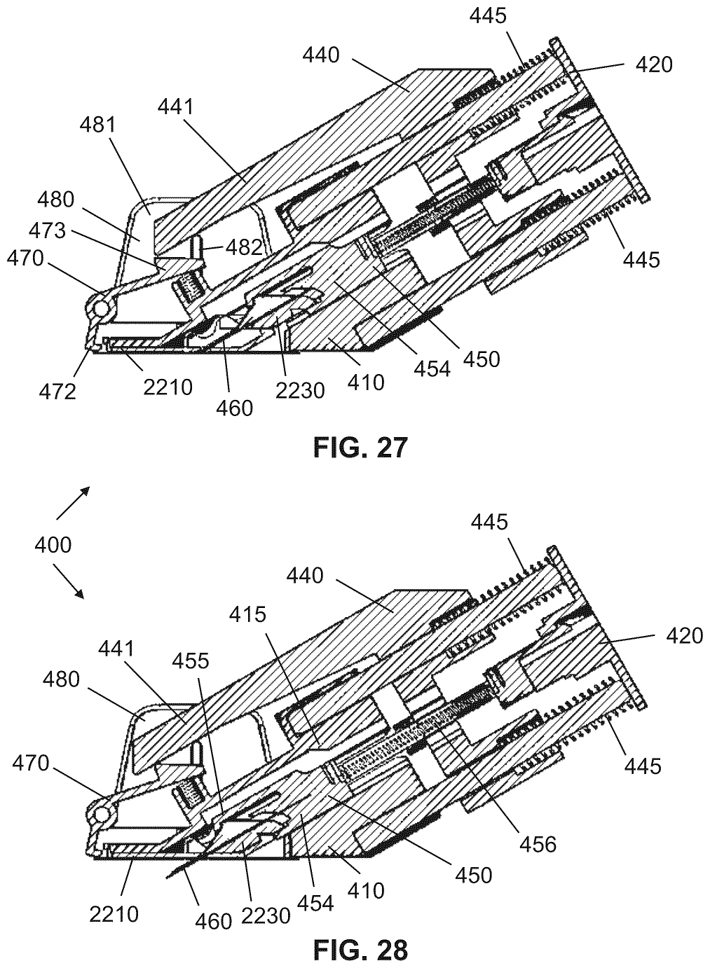

In FIG. 27, without the obstruction formed by the blocking rib 482 of the button 480, the plunger member 440 is free to advance distally, and does so via the force generated from the firing springs 445. Distal advancement of the plunger member 440 further advances the main body 454 of the needle carrier 450, along with needle 460 and sensor member 2230, by virtue of the wings 442 of the plunger member urging the arms 452 and tabs 453 of the needle carrier 450 in a distal direction as well. Similar to the third embodiment, the distal movement of the main body 454 of the needle carrier 450 causes the arms 452 to stretch out and elongate axially while compressing or constricting radially.

Remaining on FIG. 27, prior to the needle 460 reaching the patient's skin, the post 441 of the plunger member 440 hits and engages the sloped surface 473 of the latch 470, which causes the projection 472 to release from the cradle 2210, thereby releasing the hold on the cradle 2210 by the applicator assembly 400 before the needle 460 punctures the patient's skin and the analyte sensor 2231 is implanted under the surface of the patient's skin, and before the sensor member 2230 has been finally assembled together with the cradle 2210.

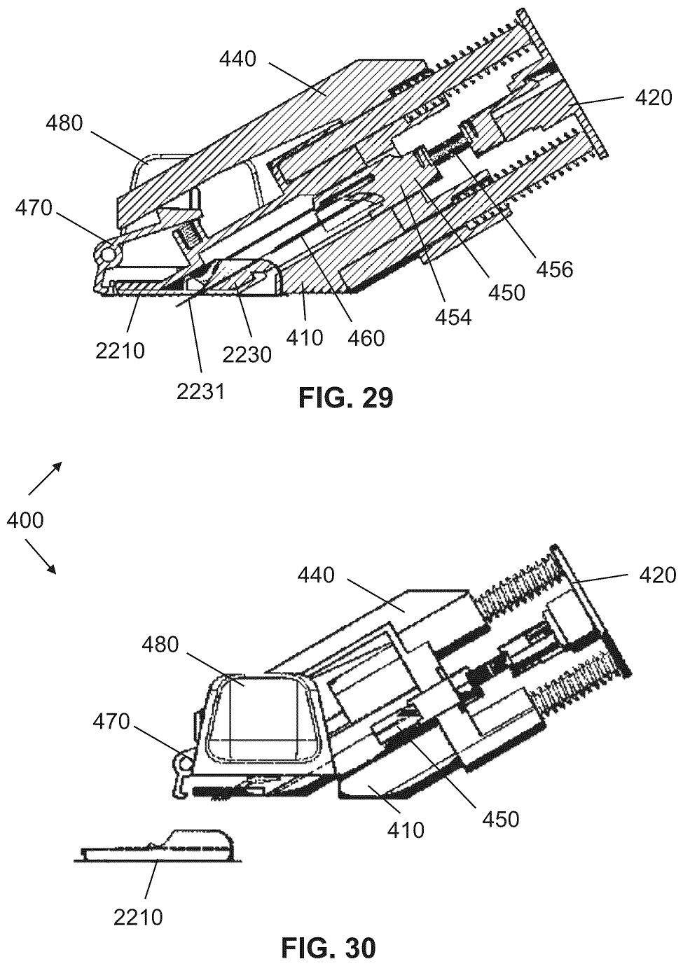

Referring now to FIG. 28, the plunger member 440 is further advanced in the distal direction by the firing springs 445 until the needle 460 punctures the skin of the patient. At this point, the sensor member 2230 latches to the cradle 2210, and the latch 455 has been advanced distally a sufficient amount so as to be clear from the stop feature 415, so that the latch 455 will detach from the sensor member 2230 automatically after the sensor member 2230 has been latched to the cradle 2210. At this point, similar to the third embodiment, the tabs 453 on the arms 452 of the needle carrier 450 have moved radially inwardly a sufficient amount so as to be able to slide inside and proximally relative to the wings 442 of the plunger member 440, so that the main body 454 of the needle carrier 450 can begin retracting relative to the other portions of the applicator assembly 400. The resiliency of the arms 452 and the extension of the spring 456 will serve to start urging the main body 454 of the needle carrier 450, along with the needle 460, in the proximal direction, while the delivered sensor member 2230 remains attached to the cradle 2210. This can be seen in FIG. 29. Furthermore, as noted above, the needle holder 450 may further include a separate snap or latching feature that holds the main body 454 of the needle holder 450 and the attached needle 460 at a sufficiently retracted position, such that there is no chance that the tip of the needle 460 extends out of the distal end of the applicator assembly 400, such that the applicator assembly 440 provides an effective sharps guard for the biohazard needle after the applicator assembly 440 has been used.

Finally, in FIG. 30, the patient pulls the applicator assembly 400 away from the assembled cradle 2210 and sensor member 2230, and can thereafter discard the applicator assembly 400. The applicator assembly 400 in some embodiments may be removed in a direction perpendicularly away from the surface of the skin of the patient, or angled in a direction opposite the direction of insertion, or either or both, or at another angle. The cradle 2210 is held in place on the patient's skin, for example, via the adhesive on the bottom side thereof, while the analyte sensor 2231 is securely held at a desired location under the surface of the patient's skin. Thereafter, in cases where a cradle is applied by the applicator assembly, a separate transmitter or other durable or reusable portion may then be attached to the cradle, both mechanically and electrically, to complete the assembly of the analyte monitor.

In addition to the embodiments that have already been described above, it is also possible to combine embodiments, e.g., different features from the various described embodiments, to provide even more different variations of applicator assemblies and/or monitors, without departing from the spirit or scope of the invention. In addition, the inventions should not be limited to the structures and/or shapes described in the embodiments above.

Unless otherwise defined, all terms (including technical and scientific terms) used herein have the same meaning as commonly understood by one of ordinary skill in the art to which the present disclosure belongs. It will be further understood that terms, such as those defined in commonly used dictionaries, should be interpreted as having a meaning that is consistent with their meaning in the context of the relevant art and/or the present disclosure, and should not be interpreted in an idealized or overly formal sense, unless expressly so defined herein.

While the subject matter of the present disclosure has been described in connection with certain embodiments, it is to be understood that the subject matter of the present disclosure is not limited to the disclosed embodiments, but, on the contrary, the present disclosure is intended to cover various modifications and equivalent arrangements included within the spirit and scope of the appended claims, and equivalents thereof.

What is claimed is:

1. An applicator assembly for applying at least a portion of an analyte monitor, the applicator assembly comprising:
a housing having a first end, an opposite second end, and a longitudinal axis extending between the first and second ends;
an engagement portion at the second end configured to releasably engage at least part of the analyte monitor;
a resilient member positioned at least partially in the housing and having at least a first portion fixed relative to the housing and a second portion movable relative to the housing in a direction substantially parallel to the longitudinal axis;
a needle movable together with the second portion of the resilient member; and
an actuator;
wherein when the actuator is actuated, the second portion of the resilient member and the needle are advanced towards the second end of the housing;
wherein when a tip of the needle is advanced to a first position located axially between the first and second ends of the housing, the engagement portion is adjusted from a first configuration where a portion of an analyte monitor connected to the applicator assembly is engaged by the engagement portion to hold the portion of the analyte monitor to the applicator assembly, to a second configuration where the engagement between the engagement portion and the portion of the analyte monitor is released; and
wherein the tip of the needle is configured to be further advanced to a second position located axially past the second end of the housing after the engagement portion is adjusted to the second configuration and the portion of the analyte monitor is released.

2. The applicator assembly of claim 1, wherein the engagement portion comprises a latch, and wherein the latch is configured to engage the portion of the analyte monitor in the first configuration and to release the engagement with the portion of the analyte monitor in the second configuration.

3. The applicator assembly of claim 1, further comprising a plunger member configured to adjust the engagement portion from the first configuration to the second configuration.

4. The applicator assembly of claim 3, wherein the plunger member is movable together with the second portion of the resilient member and the needle towards the second end of the housing.

5. The applicator assembly of claim 4, wherein when the plunger member, the second portion of the resilient member, and the needle are moved towards the second end of the housing, the plunger member adjusts the engagement portion from the first configuration to the second configuration prior to the tip of the needle reaching the second end of the housing.

6. The applicator assembly of claim 4, wherein the plunger member is configured to urge the second portion of the resilient member and the needle towards the second end of the housing.

7. The applicator assembly of claim 6, wherein when the tip of the needle is advanced to the second position, the second portion of the resilient member and the needle are configured to move back towards the first end of the housing, while an axial position of the plunger member remains the same.

8. The applicator assembly of claim 1, wherein at least part of the second end of the housing extends substantially in a plane, and wherein the longitudinal axis is arranged to be angled relative to the plane.

9. The applicator assembly of claim 8, wherein the angle is between 25° and 35°.

10. The applicator assembly of claim 1, wherein when the actuator is actuated, the tip of the needle is configured to advance continuously through the first position to the second position.

11. The applicator assembly of claim 1, wherein the actuator comprises an abutment that blocks advancement of the second portion of the resilient member and the needle prior to actuation.

12. The applicator assembly of claim 1, wherein when the portion of the analyte monitor is connected to the applicator assembly, the portion of the analyte monitor is positionable between the actuator and a desired application site, such that actuation of the actuator presses the portion of the analyte monitor against the desired application site.

13. The applicator assembly of claim 1, wherein the portion of the analyte monitor comprises a cradle or base of a two-part analyte monitor.

14. The applicator assembly of claim 1, wherein the second portion of the resilient member is further configured to connect a sensor member that is separate from the portion of the analyte monitor to the portion of the analyte monitor when the tip of the needle is advanced to the second position.

15. The applicator assembly of claim 14, wherein a sensing portion of the sensor member is configured to be positionable in the needle, such that when the tip of the needle is advanced to the second position, the needle is configured to puncture a patient's skin, and the sensor member is configured to be delivered with the needle to a position under a surface of the patient's skin.

16. The applicator assembly of claim 15, wherein the needle is configured to be retracted away from the patient's skin after the tip of the needle reaches the second position, while the sensing portion of the sensor member is configured to separate from the needle and remain at the position under the surface of the patient's skin.

17. A method of applying a portion of an analyte monitor to a patient comprising:
adhering the portion of the analyte monitor to a desired application site while the portion of the analyte monitor is connected to an applicator assembly; and
actuating the applicator assembly to advance a needle carrying at least a sensing region of a sensor member towards the desired application site;
wherein the applicator assembly is configured to release the connection between the portion of the analyte monitor from the applicator assembly prior to the tip of the needle reaching the desired application site;
wherein the tip of the needle is configured to puncture the desired application site after the connection between the portion of the analyte monitor and the applicator assembly has been released; and
wherein the sensor member is configured to be connected to and remain held by the portion of the analyte monitor at the desired application site while the needle is configured to retract away from the desired application site.

18. The method of claim 17, wherein the applicator assembly comprises a latch configured to engage the portion of the analyte monitor to connect the portion of the analyte monitor to the applicator assembly, and wherein the latch is configured to disengage from the portion of the analyte monitor when the connection between the portion of the analyte monitor and the applicator assembly is released.

19. The method of claim 18, wherein the applicator assembly further comprises a plunger member configured to be advanced together with the needle upon actuation, and wherein the plunger member is configured to disengage the latch from the portion of the analyte monitor prior to the tip of the needle reaching the desired application site.

20. The method of claim 17, wherein the actuating of the applicator assembly is configured to initiate the release of the connection between the portion of the analyte monitor from the applicator assembly.
